(19) United States
(12) Patent Application Publication
(10) Pub. No.: US 2019/0298855 A1
(43) Pub. Date: Oct. 3, 2019

(54) NANOPARTICLES WITH ACTIVE TARGETING

(71) Applicant: Cristal Delivery B.V., Maastricht (NL)

(72) Inventors: Cristianne Johanna Ferdinand RIJCKEN, Maastrict (NL); Josephus Johannes WETERINGS, Maastricht (NL); Johannes Bernardus Maria WIT, Maastricht (NL)

(73) Assignee: Cristal Delivery B.V., Maastricht (NL)

(21) Appl. No.: 15/777,391

(22) PCT Filed: Nov. 18, 2016
(86) PCT No.: PCT/NL2016/050811

§ 371 Date: May 18, 2018

(30) Foreign Application Priority Data
Nov. 20, 2015 (EP) 15195695.0

(51) Int. Cl. A61K 47/69; A61K 31/337; A61K 38/12; A61K 49/00

(52) U.S. Cl. CPC A61K 47/6935; A61K 31/337; A61K 49/0032; A61K 49/0093; A61K 38/12

(57) ABSTRACT

The present invention is directed to particle comprising a drug and a ligand, and to method of making them and use thereof. Particularly, the present invention results in the covalent entrapment of drug(s) in high amounts in polymer carriers, such as (nano)particle, microspheres and other types of polymer devices for controlled release. The polymer carriers or devices can be decorated with ligands, allowing for targeting specific tissues and/or (non-) invasive monitoring.

### CROSS-REFERENCE TO RELATED APPLICATIONS

This application is the national phase of PCT application PCT/NL2016/050811 having an international filing date of 18 Nov. 2016, which claims benefit of European patent application No. 15195695.0 filed 20 Nov. 2015. The contents of the above patent applications are incorporated by reference herein in their entirety.

The present invention relates to nanoparticles, and especially nanoparticles for the controlled release of active biological or therapeutical compounds or compositions, and specifically nanoparticles with a surface modification allowing active targeting to deliver an active ingredient to a specific part of a system to be treated. In addition, the present invention relates to a process for selectively tweaking (optimizing or tuning) nanoparticles to increase and even maximize the biological or therapeutical outcome.

More specifically, the present invention involves the selective optimizing of nanoparticle delivery systems or the selective adjustment of properties of nanoparticle delivery systems with the aim of increasing and maximizing the biological or therapeutical outcome.

The nanoparticles of the present invention are based on thermo-sensitive block copolymers. Particularly, copolymers based on PEG-b-poly(N-hydroxyalkyl methacrylamide-oligolactates) with partially methacrylated oligolactate units are preferred, but also other (meth)acrylamide esters can be used to construct the thermosensitive block, e.g. esters, and optionally (oligo)lactate esters, of HPMAm (hydroxypropyl methacrylamide) and HEMAm (hydroxyethylmethacrylamide), and N-(meth)acryloyl amino acid esters. Also preferred thermo-sensitive block copolymers are derived from monomers containing functional groups which may be modified by derivatised and underivatised methacrylate groups, such as HPMAm-lactate polymers; that is, this modification encompassing the incorporation of linker moieties.

Other types of functional thermosensitive (co)polymers, which can be used, are hydrophobically modified poly(N-hydroxyalkyl) (meth)acrylamides, copolymer compositions of N-isopropylacrylamide (NIPAAm) with monomers containing reactive functional groups (e.g., acidic acrylamides and other moieties such as N-acryloxysuccinimide) or similar copolymers of poly(alkyl) 2-oxazalines, etc.

Further preferred thermo-sensitive groups can be based on NIPAAm and/or alkyl-2-oxaxolines, which monomers may be reacted with monomers containing a reactive functional group such as (meth)acrylamides or (meth)acrylates containing hydroxyl, carboxyl, amine or succinimide groups.

Suitable thermo-sensitive polymers are described in U.S. Pat. No. 7,425,581 and in EP-A-1 776 400. Further, in WO 2010/033022 and WO2013/002636. WO2012/039602 drug-polymer matrix particles are described using such polymers. Moreover, in WO 2012/039602 biodegradable linker molecules are described that may be used in these known polymer matrix particles.

In pharmaceutical lead optimisation programs a number of parameters have to and can be selectively optimised to fine-tune the end product and its application dependent on the disease or indication to be treated.

An important aspect is the size of the nanoparticles. In respect of the present invention, this size is primarily determined by the length of the polymer chains that should form the nanoparticles optionally in combination with the use of specific cross-linking moieties.

In practice, when a disease or indication is to be treated, the skilled person knows or has to determine which part of a system to be treated needs to be or can be targeted; for example, which receptor or receptors is/are overexpressed and which can be targeted.

After selection of the systemic target (such as a receptor), a corresponding ligand or other targeting or homing device (such as an antibody or a nanobody, a peptide or another small molecule selective for binding to a specific target) is to be selected and has to be coupled to the outer surface of the nanoparticles of the present invention. In some embodiments, it is preferred to have more than one ligand or other targeting or homing device present on the outer surface of the nanoparticles.

After the selection of the ligand, the linking method to attach the ligand to the nanoparticle has to be selected. An important consideration is whether the ligand should remain stably conjugated or potentially be cleaved off in time.

Once the ligand and conjugation chemistry are determined, a range of nanoparticles having different degrees of ligand attachment is to be defined. For, one has to optimize the number of ligands on a nanoparticle based on early preclinical studies. That is, the pharmacokinetics profile (herein-after: the “PK profile”) has to be determined versus the efficacy of the binding. The option to conjugate a targetomg compound stably to the surface to the nanoparticles can also be used to conjugate therapeutic compound or a dye, radioactive agent or similar to enable (non)invasive imaging, and to get thereby insight into the pharmacokinetic and biodistribution profile of nanoparticles, and thereby also in the potentially different profiles of nanoparticles with variable pharmaceutical features as size, degradation profile etc.

Once the vehicle, the nanoparticle system, is defined, one can select the active ingredient to be delivered to the target. Often, for an efficacious treatment, one tries to select the most potent active ingredient, such as a drug. However, it is also possible to load the vehicle with more than one type of active ingredient.

Dependent on the indication, the choice made for the active ingredient and consequently the dosage regimen, and dependent on where the active ingredient(s) is (are) to be released, the degradability of the nanoparticles and the drug linking chemistry must be determined, controlled and adjusted. This should lead to the desired release profile from the loaded nanoparticle system, and can vary between fast release to sustained and prolonged release.

For example, based on the application frequency of the nanoparticles, the degradability profile of the nanoparticles has to be selected. In for instance the chronic treatment of cancer, it can be imagined that one wishes to have a rather fast exposure to the release drug molecule (and thus a fast degradation, but for other applications one may wish to have a slow degradation. Dependent on the availability of the active ingredients, also the degradation of the nanoparticle system needs to be tuned.

In the prior art, for many nanoparticle systems the adjustment of one property immediately results in negative effects on another property. It is an aim of the present invention to come to a system wherein a flexible adjustment of selective parameters results in optimized properties for the final system.

Also to the surprise of the present inventors, the nanoparticle system that forms the basis of the technology to which the present invention relates provides the freedom to create tailor-made systems. That is, the nanoparticle system based on the polymers identified above should lead to a delivery system with improved efficacy and tolerability of the active compounds entrapped therein. This should be effected by an improved disposition in the system, or part of the system, to be treated; by an active targeting at the site needing the treatment, whether this is at tissue, cellular or molecular level; by a prolonged circulation; and by a tuneable and timely release of the active ingredients. The opportunity to non-invasively (via conjugation of a label to the surface) follow the nanoparticles with variable pharmaceutical features will thereby enable fast insight into the biological profile, and thereby facilitate in selection the best options.

The requirement adjustment steps in the system of the present invention will be elaborated in the following part.

Incidently, WO 2014/142653 describes that it appears that the uptake of nanoparticles in target cells is largely size and composition dependent, thus requiring full control of the size and composition. For many nanoparticles, and methods of production in the prior art however it is not possible to control the size and/or composition. Indeed, most of the particles of the prior art show a large distribution in size, thereby rendering a particle based on the particles heterogeneous, or requiring further purification. Furthermore for controlled release purposes there has to be a tight control over the encapsulation of drug, and/or attachment of the ligand to ensure batch to batch reproducibility. Most of the nanoparticles in the prior art do not enable such a tight control, and are therefore not suitable to generate a robust therapeutic response.

WO2010/138193 is directed to compositions of synthetic nanocarriers that may target sites of action in cells, such as drug presenting cells and comprise immunomodulatory agents that dissociate from the synthetic nanocarriers in a pH sensitive manner. The synthetic nanocarriers of WO2010/138193 are preferentially taken up by Antigen-Presenting Cells (APCs). Upon being taken up by the APC, the synthetic nanocarriers are presumed to be endocytosed into an endosomal/lysosomal compartment where the pH becomes more acidic, as opposed to the neutral pH outside the cells. Under these conditions, the immunomodulatory agent exhibits a pH sensitive dissociation from the synthetic nanocarrier and is released from the synthetic nanocarrier. The immunomodulatory agent is then free to interact with receptors associated with the endosome/lysosome and stimulate a desired immune response. However WO2010/138193 does not discloses cross-linking of the polymers when the immunomodulatory agent is present. There is no disclosure of a system wherein the immunomodulatory agent is covalently entrapped into the nanoparticle. In WO2010/138193 particles are made by first conjugating the immunomodulatory agent to the polymer and then make nanoparticles of the immunomodulatory agent-polymer complex. The system of WO2010/138193 thus requires different routes for conjugation for each different immunomodulatory agent.

US2009/011993 is directed to particles that deliver active agents such as vaccines, immune modulatory agents and/or drugs that target antigen presenting cells. US2009/011993 discloses a new type of hydrophobic polymers comprising ketal groups in the polymer backbone wherein the ketal groups are arranged in a way such that both oxygen atoms are located in the polymer backbone. US2009/011993 discloses the use of an external crosslinking agent to cross-link the polymers to the immune modulatory agents, and does not disclose a crosslinking step of the polymers in the presence of immune modulatory agents.

Rijcken et al. (Biomaterials 2007; 28(36): 5581-5593) describes core-crosslinked polymeric micelles based on (100%) mPEG5000 and N-(2-hydroxyethyl)methacrylamide)-oligolactates and studies their properties. These micelles do not contain a covalently entrapped drug nor is there the possibility to conjugate a ligand to their surface.

EP 1776400 describes degradable thermosensitive polymeric micelles. The micelles contain a non-covalently entrapped drug, such as paclitaxel. Covalent attachment of a targeting or imaging ligand on the surface of the micelles is not described.

Talelli et al (Biomaterials 2010; 31(30): 7797-7804) describes cross-linked polymeric micelles with entrapped doxorubicin. The micelles do not contain a targeting or imaging ligand on their surface, and the polymers do not contain azide or alkyne groups that allow attachment to such ligand by click chemistry.

For most systems of the prior art, either being a vaccine or therapeutic system the particles and conjugation need an optimisation for each different active agent, such as an immunomodulatory agent or drug. This requires extensive research for each new particle with other active ingredients, and creates differences between the different active ingredients. The optimum formulation optionally depends on the type of response required and the intended route of administration. Various formulation aspects, such as particles size, targeting ligand, etc., are optionally adjusted based on the selected administration route. The nanoparticles described in the prior art may be suitable for one particular drug/active ingredient and for one particular route of administration, but are often unsuitable for another drug and/or other route of administration. Thus for different treatment routes, each time a different nanoparticle has to be developed.

It is an object of the present invention to provide a particle that is easily adjustable for different purposes. Further, another object of the present invention is to provide a particle wherein the particles have a narrow size distribution. Yet another object of the invention is to provide a particle that may accommodate different drugs, active compounds and/or ligands, e.g. both hydrophilic and hydrophobic compounds and over a large size range. Moreover, another object of the invention is to provide a particle wherein the release of the drug or active compound can be controlled, e.g. under physiological conditions or selectively at the target site. Another object of the invention is to provide a particle wherein each entrapped drug or active compound and/or ligand has its own unique release profile. Even another object of the present invention is to provide a particle that comprises ligands covalently attached to the particle, e.g. to its surface. The ligand may direct the particle of the present invention to the cells or site of interest, such as APCs or tumour cells. Alternatively or additionally, the ligand may be used in (non-)invasive imaging. Alternatively or additionally, the ligand may be a therapeutic compound such as a peptide. Yet another object of the invention is to provide a method for producing the particle that is safe and/or non-destructive for the drug entrapped inside the particle. Also an object of the invention is to have control over the degradation of the particles for release of the drug.

The present invention provides a particle that meets one or more of the above mentioned objects.

### SUMMARY OF THE INVENTION

In a first aspect, the present invention provides a particle comprising a drug and a ligand wherein the particle is obtainable by a method comprising the steps of:

(i) mixing a drug comprising a reactive moiety with an aqueous solution or dispersion comprising polymer chains, at least part of these polymer chains comprising at least one azide group or at least one alkyne group, the polymer chains comprising at least one reactive moiety, capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture to cross-linking forming a polymer matrix under such conditions that simultaneous with the formation of the polymer matrix the drug is entrapped in this polymer matrix, that is in the polymeric network formed, to form a drug loaded particle;

(iva) reacting said drug entrapped particle with a ligand comprising at least one alkyne group when the polymer chain comprises an azide group or

(ivb) reacting said drug entrapped particle with a ligand comprising at least one azide group when the polymer chain comprises an alkyne group, such that the azide group reacts with the alkyne group to form a triazole.

The end result of such method is accordingly a particle that is preferably truly a single macromolecule since all components (i.e. drug, ligand and polymer chains) are covalenty linked to each other.

Polymers or ligand may be derivatised by an azide group by methods known by the skilled person. In principle this reaction of the azide group with the alkyne moiety can be achieved using all known methods including the use of heating and the use of (metal) catalysts for example by use of a copper catalyst.

In this method of the invention, reference is made to “drug”. However, this term should not be interpreted in a limiting sense. It encompasses all kind of active ingredients having an intended effect on or in the system to be treated.

In a further aspect, the present invention provides a particle comprising a ligand wherein the particle is obtainable by a method comprising the steps of:

(i) subjecting an aqueous solution or dispersion comprising polymer chains, at least part of these polymer chains comprising at least one azide group or at least one alkyne group, the polymer chains further being capable of cross-linking intra- or intermolecularly to conditions wherein the polymers self-assemble into particles;

(ii) subjecting the particles to cross-linking forming a polymer matrix;

(iiia) reacting said particles with a ligand comprising at least one alkyne group when the polymer chain comprises an azide group, or

(iiib) reacting said particles with a ligand comprising at least one azide group when the polymer chain comprises an alkyne group,

such that the azide group reacts with the alkyne group to form a triazole bond.

For the present inventors, it was highly unexpected that the introduction of the azide or alkyne groups on the surface of the nanoparticles or on the ligand and the subsequent reaction by click chemistry between azide and alkyne group did not essentially affect the size of the nanoparticles; that is, the surface of the nanoparticles is not or only minimally changed. Moreover, the conversion of the azide or alkyne groups to links with the ligands turned out to be very high, where generally only a limited number of ligands already provide a suitable targeting possibility. Hence, the present invention allows full control of the extent of conjugation at the nanoparticle surface. The conjugation of the ligand to the polymer may be monitored by NMR. The reaction between the azide and alkyne forms a triazole group and may be shown by means of ¹⁵N NMR. In addition, the drug release profile of the particles was also not changed, including no increase in burst release. This thus shows that for the present particles it is possible to modify or optimise one particular property of the particle without affecting the other properties. A further benefit is that the azide-alkyne reaction can be easily carried out without a catalyst and without elevated temperatures. Examples of ligands that can be covalently attached to the surface of the particles are therapeutic ligands, targeting ligands and/or imaging ligands.

In another aspect, the present invention provides a particle comprising a drug wherein the drug is present in an amount of at least 10 wt %, the particle is obtainable by a method comprising the steps of:

(i) mixing a drug comprising a reactive moiety with an aqueous solution or dispersion comprising polymer chains comprising at least one reactive moiety, capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture to cross-linking forming a polymer matrix under such conditions that simultaneous with the formation of the polymer matrix the drug is entrapped in this polymer matrix, that is in the polymeric network formed, to form a drug loaded particle with a loading capacity of at least 10%.

According to another unexpected result, it was found that the amount of drug to be entrapped in the nanoparticles prepared according to the invention is much higher than expected. The amount of drugs entrapped can be expressed as loading capacity (LC) which is the weight of the drug entrapped divided by the weight of the particle expressed in %. The loading capacity of the particle of the invention is surprisingly high. It was found that the loading capacity may be at least 10%, or even at least 15%, at least 20%, at least 25%, at least 30%.

It was also found that the drug entrapment efficiency of the particles of the present invention is surprisingly high. The drug entrapment efficiency is the weight of the drug actually entrapped divided by the weight of the drug fed to the particle expressed in %. For the particle of the invention, the drug entrapment efficiency may be at least 30% or even at least 40%, at least 50%, at least 60%, at least 70% and even at least 80% and even more than 90 wt. %. Surprisingly the high loading capacity and drug entrapment capacity is also found with small particles. For example particles as small as 20-200 nm may still exhibit the high loading capacity and/or drug entrapment efficiency, even particles as small as 25-100 nm may still exhibit the high loading capacity a drug entrapment efficiency or even particles as small as 30-75 nm may still exhibit the high loading capacity a drug entrapment efficiency, still even particles as small as 35-65 nm may still exhibit the high loading capacity a drug entrapment efficiency.

It was also found that particles with a loading capacity of at least 10% are not significantly larger than particles with a loading capacity of less than 10%. In addition, the drug release profile was also not significantly changed in particles with a high drug loading capacity.

The polymer matrix is the polymeric network formed. The drug is thus entrapped in the polymer matrix, in the polymer network formed by the crosslinking. The drug cross-links to the polymer chains and thus forms also covalent part of the polymer matrix.

In a preferred embodiment, after formation of drug entrapped particles with a loading capacity of at least 10%, ligands may be conjugated to the surface of the drug loaded particle. Optionally the ligand is conjugated to the particle via azide-alkyne cycloaddition. An azide group may be present on the polymer and an alkyne group may be present on the ligand. I.e. at least part of the polymer chains comprise an azide group and the ligand comprises an alkyne group. In addition, an alkyne group may be present on the polymer and an azide group may be present on the ligand. I.e. at least part of the polymer chains comprise an alkyne group and the ligand comprises an azide group. In a preferred embodiment, at least part of the polymer chains comprise an azide group and the ligand comprises an alkyne group.

In a further aspect, the present invention provides a method to produce a particle comprising a drug and a ligand, said method comprising the steps of:

i) mixing a drug comprising a reactive moiety with an aqueous solution or dispersion comprising polymer chains, at least part of these polymer chains comprising at least one azide group or at least one alkyne group, the polymer chains comprising at least one reactive moiety capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture from step (ii) to cross-linking forming a polymer matrix under such conditions that simultaneous with the formation of the polymer matrix the drug is entrapped in this polymer matrix, that is in the polymeric network formed, to form a drug loaded particle;

(iva) reacting said drug entrapped particle with a ligand comprising at least one alkyne group when the polymer comprises an azide group or

(ivb) reacting said drug entrapped particle with a ligand comprising at least one azide group when the polymer comprises an alkyne group such that the azide group reacts with the alkyne group to form a triazole.

In a further aspect, the present invention provides a method to produce a particle comprising a ligand, said method comprising the steps of:

(i) subjecting an aqueous solution or dispersion comprising polymer chains, at least part of these polymer chains comprising at least one azide group or at least one alkyne group, the polymer chains further being capable of cross-linking intra- or intermolecularly to conditions wherein the polymers self-assemble into particles;

(ii) subjecting the particles to cross-linking forming a polymer matrix;

(iiia) reacting said particles with a ligand comprising at least one alkyne group when the polymer chain comprises an azide group, or

(iiib) reacting said particles with a ligand comprising at least one azide group when the polymer chain comprises an alkyne group, such that the azide group reacts with the alkyne group to form a triazole bond.

In a further aspect, the present invention provides a method to produce a particle comprising a drug wherein the drug is present in an amount of at least 10 wt %, said method comprising the steps of:

i) mixing a drug comprising a reactive moiety with an aqueous solution or dispersion comprising polymer chains comprising at least one reactive moiety capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture from step (ii) to cross-linking forming a polymer matrix under such conditions that simultaneous with the formation of the polymer matrix the drug is entrapped in this polymer matrix, that is in the polymeric network formed, to form a drug loaded particle with a loading capacity of at least 10%.

In a preferred embodiment, a ligand is conjugated to the surface of the drug loaded particle with a loading capacity of at least 10%. Optionally the ligand is conjugated to the particle via azide-alkyne cycloaddition. An azide group may be present on the polymer and an alkyne group may be present on the ligand. I.e. at least part of the polymer chains comprise an azide group and the ligand comprises an alkyne group. In addition, an alkyne group may be present on the polymer and an azide group may be present on the ligand. I.e. at least part of the polymer chains comprise an alkyne group and the ligand comprises an azide group. In a preferred embodiment, at least part of the polymer chains comprise an azide group and the ligand comprises an alkyne group.

In a further aspect of the invention, the present invention provides a method to produce a particle comprising a drug and a ligand, said method comprising the steps of:

(i) providing an aqueous solution or dispersion comprising polymer chains comprising polymer chains, at least part of these polymer chains comprising at least one azide group or at least one alkyne group, the polymer chains comprising at least one reactive moiety, capable of reacting with the reactive moiety of the drug, the polymer chains further being capable of cross-linking intra- or intermolecularly; and

(ii) subjecting this mixture to conditions whereby the polymers self-assemble into particles, and,

(iii) mixing the particle from step (ii) with a solution comprising a drug such that the drug is encapsulated in the particle, and;

(iv) subjecting the particle mixture from step (iii) to cross-linking forming a polymer matrix under such conditions that simultaneous with the formation of the polymer matrix the drug is entrapped in this polymer matrix, that is in the polymeric network formed, to form a drug loaded particle;

(va) reacting said drug entrapped particle with a ligand comprising at least one alkyne group when the polymer chain comprises an azide group, or

(vb) reacting said drug entrapped particle with a ligand comprising at least one azide group when the polymer chain comprises an alkyne group,

such that the azide group reacts with the alkyne group to form a triazole.

In a further aspect of the invention, the present invention provides a method to produce a particle comprising a drug, wherein the drug is present in said particle in an amount of at least 10 wt %, said method comprising the steps of:

(i) providing an aqueous solution or dispersion comprising polymer chains comprising and at least one reactive moiety, capable of reacting with the reactive moiety of the drug, the polymer chains further being capable of cross-linking intra- or intermolecularly; and

(ii) subjecting this mixture to conditions whereby the polymers self-assemble into particles, and,

(iii) mixing the particle from step (ii) with a solution comprising a drug such that the drug is encapsulated in the particle, and;

(iv) subjecting the particle mixture from step (iii) to cross-linking forming a polymer matrix under such conditions that simultaneous with the formation of the polymer matrix the drug is entrapped in this polymer matrix, that is in the polymeric network formed, to form a drug loaded particle with a loading capacity of at least 10%;

After crosslinking, a ligand may be conjugated to the surface of the generated particles. In a preferred embodiment, a ligand is conjugated to the surface of the drug loaded particle with a loading capacity of at least 10%. Optionally the ligand is conjugated to the particle via azide-alkyne cycloaddition. An azide group may be present on the polymer and an alkyne group may be present on the ligand. I.e. at least part of the polymer chains comprise an azide group and the ligand comprises an alkyne group. In addition, an alkyne group may be present on the polymer and an azide group may be present on the ligand. I.e. at least part of the polymer chains comprise an alkyne group and the ligand comprises an azide group. In a preferred embodiment, at least part of the polymer chains comprise an azide group and the ligand comprises an alkyne group.

In a further aspect of the invention, the present invention provides a particle comprising a drug and a ligand wherein the particle is obtainable by a method comprising the steps of:

(i) providing an aqueous solution or dispersion comprising polymer chains comprising polymer chains, at least part of these polymer chains comprising at least one azide group or at least one alkyne group, the polymer chains comprising at least one reactive moiety, capable of reacting with the reactive moiety of the drug, the polymer chains further being capable of cross-linking intra- or intermolecularly; and

(ii) subjecting this mixture to conditions whereby the polymers self-assemble into particles, and,

(iii) mixing the particle from step (ii) with a solution comprising a drug such that the drug is encapsulated in the particle, and;

(iv) subjecting the particle mixture from step (iii) to cross-linking forming a polymer matrix under such conditions that simultaneous with the formation of the polymer matrix the drug is entrapped in this polymer matrix, that is in the polymeric network formed, to form a drug loaded particle;

(va) reacting said drug entrapped particle with a ligand comprising at least one alkyne group when the polymer chain comprises an azide group, or

(vb) reacting said drug entrapped particle with a ligand comprising at least one azide group when the polymer chain comprises an alkyne group,

such that the azide group reacts with the alkyne group to form a triazole.

In any aspect of the invention and/or embodiment thereof, the reaction of azide and alkyne is performed without a catalyst. In any aspect of the invention and/or embodiment thereof the reaction of azide and alkyne is performed without at room temperature In any aspect of the invention and/or embodiment thereof the reaction of azide and alkyne is performed without a catalyst and at room temperature.

In any aspect of the invention and/or embodiment thereof, 1 out of 1500 polymer chains are derivatised by an azide group, optionally 2 out of 1500, optionally 5 out of 1500, optionally 7 out of 1500, optionally 10 out of 1500, optionally 15 out of 1500, optionally 20 out of 1500, optionally 25 out of 1500, optionally 30 out of 1500, optionally 35 out of 1500, optionally 40 out of 1500, optionally 45 out of 1500, optionally 50 out of 1500, optionally 55 out of 1500, optionally 60 out of 1500, optionally 65 out of 1500, optionally 70 out of 1500, optionally 75 out of 1500, optionally 100 out of 1500, optionally 150 out of 1500, optionally 200 out of 1500, optionally 250 out of 1500, optionally 300 out of 1500, optionally 400 out of 1500 optionally 500 out of 1500, optionally 600 out of 1500, optionally 750 out of 1500, optionally 900 out of 1500, optionally 1000 out of 1500, optionally 1200 out of 1500, optionally 1300 out of 1500, optionally 1400 out of 1500 polymer chains are derivatised by an azide group.

In any aspect of the invention and/or embodiment thereof about 0.01% to about 100% of the polymer chains is derivatised by an azide group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the polymer chains is derivatised by an azide group.

In any aspect of the invention and/or embodiment thereof about 0.01% to about 100% of the ligand is derivatised by an azide group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the ligand is derivatised by an azide group.

In any aspect of the invention and/or embodiment thereof 1 out of 1500 polymer chains are derivatised by an alkyne group, optionally 2 out of 1500, optionally 5 out of 1500, optionally 7 out of 1500, optionally 10 out of 1500, optionally 15 out of 1500, optionally 20 out of 1500, optionally 25 out of 1500, optionally 30 out of 1500, optionally 35 out of 1500, optionally 40 out of 1500, optionally 45 out of 1500, optionally 50 out of 1500, optionally 55 out of 1500, optionally 60 out of 1500, optionally 65 out of 1500, optionally 70 out of 1500, optionally 75 out of 1500, optionally 100 out of 1500, optionally 150 out of 1500, optionally 200 out of 1500, optionally 250 out of 1500, optionally 300 out of 1500, optionally 400 out of 1500 optionally 500 out of 1500, optionally 600 out of 1500, optionally 750 out of 1500, optionally 900 out of 1500, optionally 1000 out of 1500, optionally 1200 out of 1500, optionally 1300 out of 1500, optionally 1400 out of 1500 polymer chains are derivatised by an alkyne group.

In any aspect of the invention and/or embodiment thereof about 0.01% to about 100% of the polymer chains is derivatised by an alkyne group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the polymer chains is derivatised by an alkyne group.

In any aspect of the invention and/or embodiment thereof about 0.01% to about 100% of the ligand is derivatised by an alkyne group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the ligand is derivatised by an alkyne group.

Suitably the polymer chains comprise reactive moieties that do not react in the cross-linking step and may be used to link a ligand to the surface of the formed and cross-linked particle. Alternatively, after the crosslinking, additional linking groups are attached to the surface of the generated cross-linked particles that may further react to conjugate a ligand to the surface of the cross-linked particle. Also reactive moieties on the polymer chains may be blocked during the cross-linking step and deblocked when the polymer network has formed. These deblocked reactive moieties may then be reacted with a ligand and/or a drug and/or an adjuvant such that these will be attached to the surface of the cross-linked particle. In addition, the drug may comprise a reactive moiety that is able to conjugate to the polymer chains of the cross-linked particles.

The method of the invention may also be used to introduce a radio label or other type of label on the particle. The particle is then or also suitable for use in diagnosis or to monitor the therapeutic activity of the particle.

In another aspect, the invention is related to a particle according to the invention for use as a medicine, preferably wherein said particle is a drug entrapped particle and/or comprises a therapeutic ligand conjugated to its surface.

Yet another aspect of the invention is related to a method of treatment using the particle of the invention, preferably wherein said particle is a drug entrapped particle and/or comprises a therapeutic ligand conjugated to its surface.

In another aspect, the invention is related to a use of a particle according to the invention wherein said particle comprises an imaging label attached to the surface as a diagnostic, such as a companion diagnostic.

In aspects and/or embodiments of the invention, the polymer optionally comprises at least one reactive moiety per polymer chain and at least part of these polymer chains comprise at least one azide group or at least one alkyne per polymer chain. Also in aspects and/or embodiments of the invention the drug comprises at least one reactive moiety. Also in aspects and/or embodiments of the invention the ligand comprises at least one alkyne group or one azide group. In a preferred embodiment, at least part of the polymer chains comprise an azide group and the ligand comprises an alkyne group.

Advantageously the drug is covalently entrapped in the polymer matrix during the cross-linking step. Advantageously the drug comprises a reactive moiety and polymer chains comprise also a reactive moiety capable of reacting with the reactive moiety of the drug to allow covalent attachments between the drug and the polymer chains. During the cross-linking step, the polymers form a network together with the drug, which form the polymer-drug matrix in which the drug is entrapped. After formation of the particle, ligands comprising at least one alkyne group or at least one azide are reacted with the particle with the entrapped drug under conditions that allow the azide group to react with the alkyne group to form a stable triazole bond. The reaction of the azide with the alkyne is very mild and does not effect the drug or the drug-polymer binding, i.e. induces hardly any or no premature drug release and/or nanoparticle degradation.

The nanoparticles of the present invention are based on copolymers that self-assemble in aqueous media into micellar structures. In accordance with the present invention, nanoparticles can be prepared using varying particle sizes. This is primarily effected by altering the molecular weight of the block copolymers that build the nanoparticles, and secondarily also the type and density of the cross-linkers have an effect. This will be illustrated for PEG-b-poly[N-(2-hydroxypropyl)methacylamide-lactate] (mPEG-b-p(HPMAmLacₙ) block copolymers, but can be generalized to other types of block copolymers of the types mentioned herein-above.

Hence, the size of these nanoparticles can be adjusted by using block copolymers having a varying polymer length which block copolymers may be partially derivatised. Typically, polymer chains used in the methods and particles of the invention typically have a molecular weight of 10.000 to 30.000, but smaller and larger polymer chains can also be used.)

Optionally, the block copolymers used have a fixed hydrophilic block, such as a block of PEG (for example a monomethoxy poly(ethylene glycol); mPEG), e.g. a PEG having an Mₙ of about 5000 g/mol, but higher and lower Mₙ values will work as well; and a varying thermosensitive block. As mentioned herein-above, the present invention makes use of the fact that part of the nanoparticle forming polymer chains comprise an azide group or an alkyne group. The azide or alkyne group is preferably attached to PEG in the polymer chains used in the methods and particles of the inventions. Such an azide group or alkyne group may be introduced by starting from an azide-PEG-OH molecule or alkyne-PEG-OH molecule respectively and convert this in an initiator instead of the mPEG-OH. In the present invention, optionally between 1 and 30 wt. %, more optionally between 2 and 15 wt. %, and most optionally between 3 and 10 wt. %, optionally between 4 and 8 wt. %, such as up to 5 wt. % of the block copolymer chains will contain an azide group or an alkyne group.

These block copolymers containing a fixed hydrophilic block of monomethoxy polyethylene glycol) and a varying thermosensitive block composed of a random copolymer of HPMAmLac₁ and HPMAmLac₂ were synthesized by free radical polymerization using (mPEG₅₀₀₀)₂-ABCPA as initiatior (in addition to the above referred to azide-PEG initiator). This as described in Rijcken et al., Biomaterials 28 (2007) 5581-5593 and in Neradovic et al., Macomolecules 34 (2001), 7589-7591. The feed molar ratio of monomer/initiator was varied between 20 and 300 to obtain a set of block copolymers of different molecular weights. By derivatizing the terminal hydroxyl groups of the lactate side chains with different cross-linking moieties, such as with methacrylic acid or with 2-(2-(methacryloyloxy) ethylsulphinyl)acetic acid-pivaloyl or other cross-linking moieties, the sizes of the nanoparticles can be fine-tuned.

In general, particles are classified according to diameter. Coarse particles cover a range between 10,000 and 2,500 nanometers. Fine particles, such as microparticles are sized between 2,500 and 100 nanometers. Ultrafine particles, such as nanoparticles are sized between 1 and 100 nanometers. For the present invention, nanoparticles may range in size between 0.1 and 1000 nanometer, optionally between 1 and 500 nanometer, more optionally between 5 and 250 nanometer, more optionally between 10 and 200 nanometer, and more optionally between 30 and 150 nanometer. The size may influence the ability to be taken up by target cells. Generally virus-sized particles in the size range of 20 to 200 nm are usually taken up by endocytosis, resulting in a cellular-based immune response, whereas particles with sizes between 500 nm and 5 micron are mainly taken up by phagocytosis and/or macro-pinocytosis and are more likely to promote a humoral immune response. Specific cells usually have an upper and lower limit size for particles that may be taken up. Alternatively, if one wishes that certain cells do not take up the particles of the invention, a skilled person may choose for a size that is outside the range for these cells. The particles of the present invention may be tuned to a desired size, enabling to target specific cells. In addition, the particles made by the methods of the invention have a narrow distribution so that a large part of the particles have the desired particle size and thus can target the desired cells.

In a preferred embodiment, the particles of the present invention have a very narrow size distribution, meaning that the larger part of the particles have the same size. Optionally the particles have a polydispersity index (DPI) of less than 0.5, more optionally less than 0.4, even more optionally less than 0.3, more optionally less than 0.2 and most optionally less than 0.1, or even less than 0.05.

### DETAILED DESCRIPTION

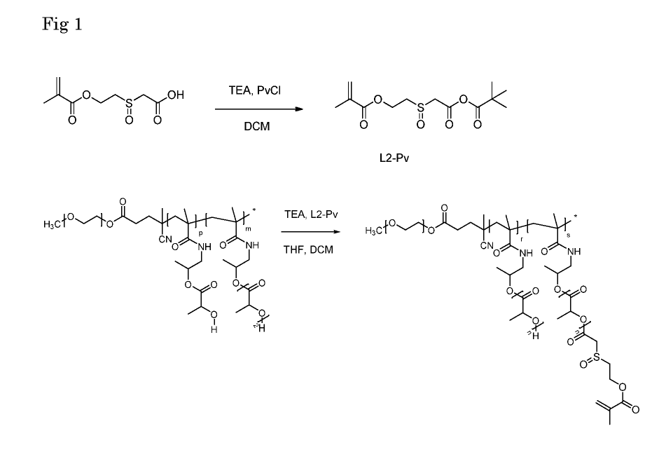

FIG. 1. Derivatisation of block copolymer mPEG₅₀₀₀-b-pHPMAmLacₙ with L2 (p and m are the numbers of HPMAmLac₁ and HPMAmLac₂ units present in the non-derivatised block copolymer, respectively; r and s are the numbers of non-derivatised and L2-derivatized HPMAmLacₙ (n=1 or 2) units present in the derivatised block copolymer, respectively)

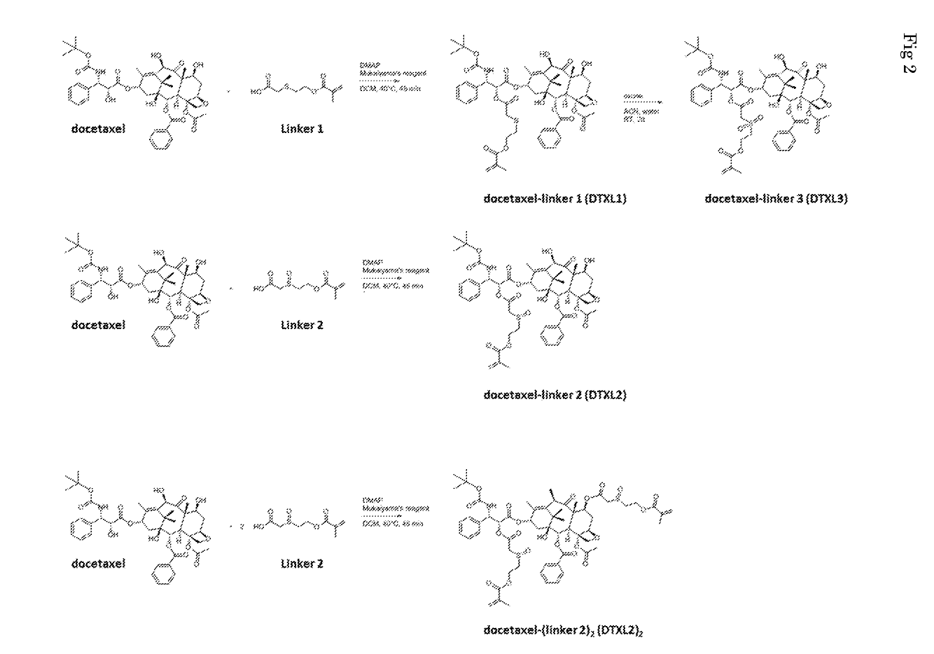

FIG. 2: Synthesis scheme of various DTX derivatives.

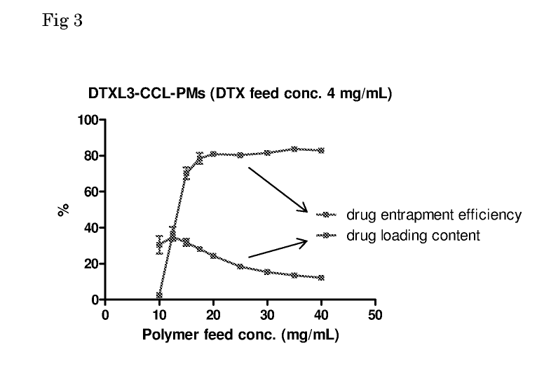

FIG. 3: Loading capacity and drug entrapment efficiency at a drug feed of about 4 mg/ml.

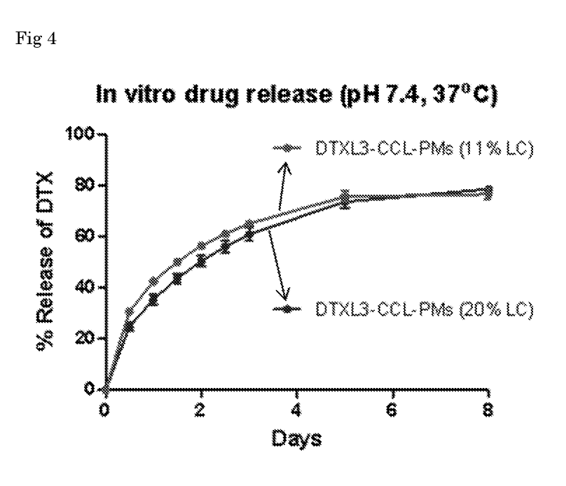

FIG. 4: drug release of particle with different loading capacity.

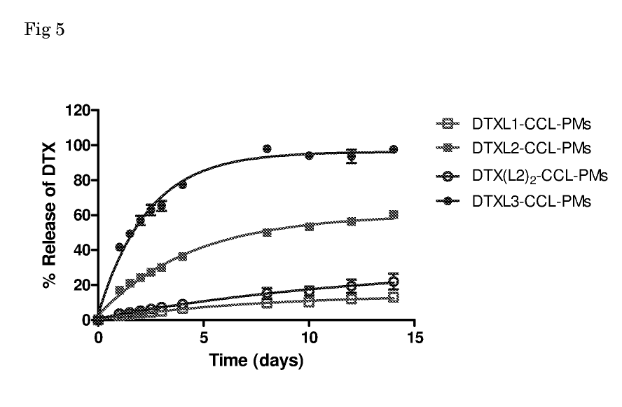

FIG. 5. In vitro release of DTX from DTX-entrapped CCL-PMs under physiological conditions (pH 7.4, 37° C.). Data are expressed as the mean±SD (n=3).

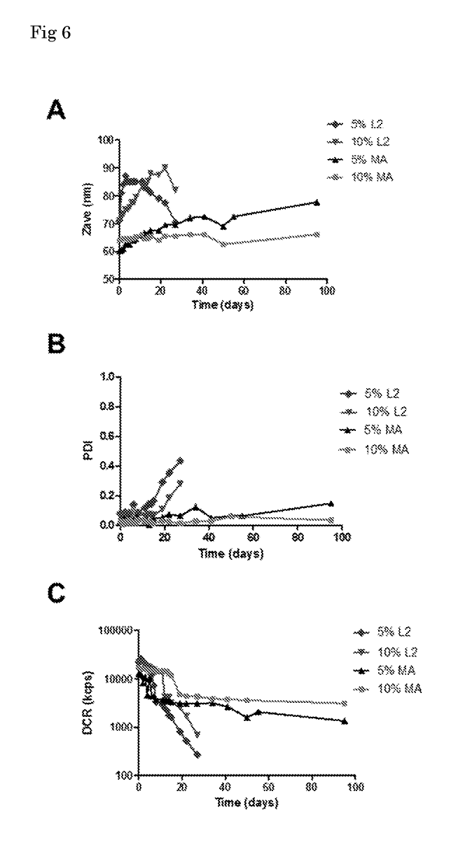

FIG. 6. Degradation characteristics of empty CCL-PMs under physiological conditions (pH 7.4, 37° C.). (A) Z-average particle size diameter; (B) polydispersity index and (C) derived count rate. Data are expressed as the mean±SD (n=3).

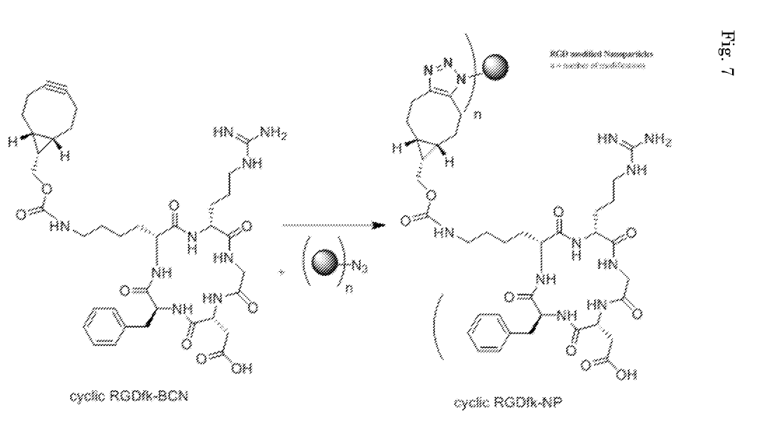

FIG. 7: Schedule indicating the formation of triazole from a cycloaddition of an alkyne derivatised ligand and an azide derivatised nanoparticle.

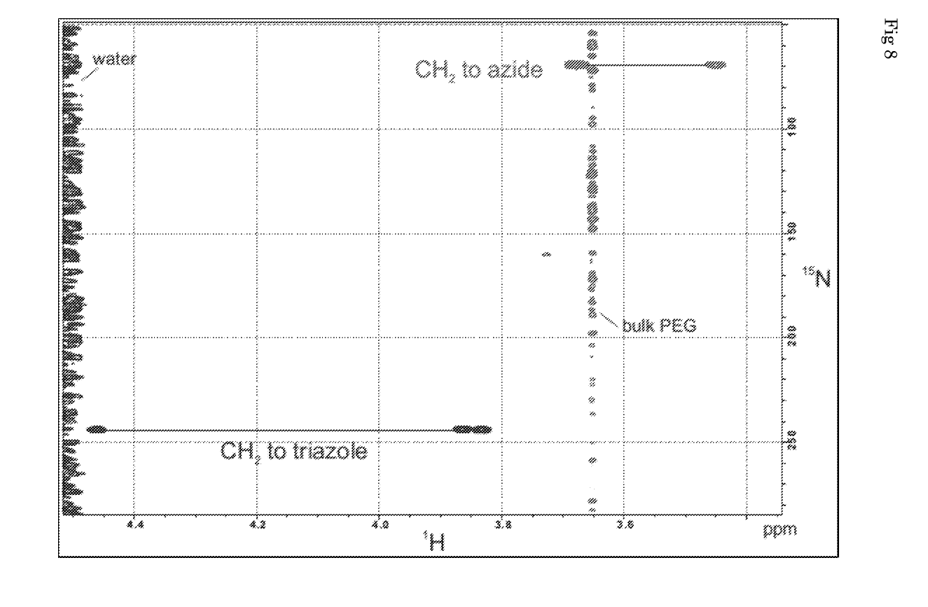

FIG. 8: NMR spectrum showing the formation of a triazole bond in RGD CriPec empty.

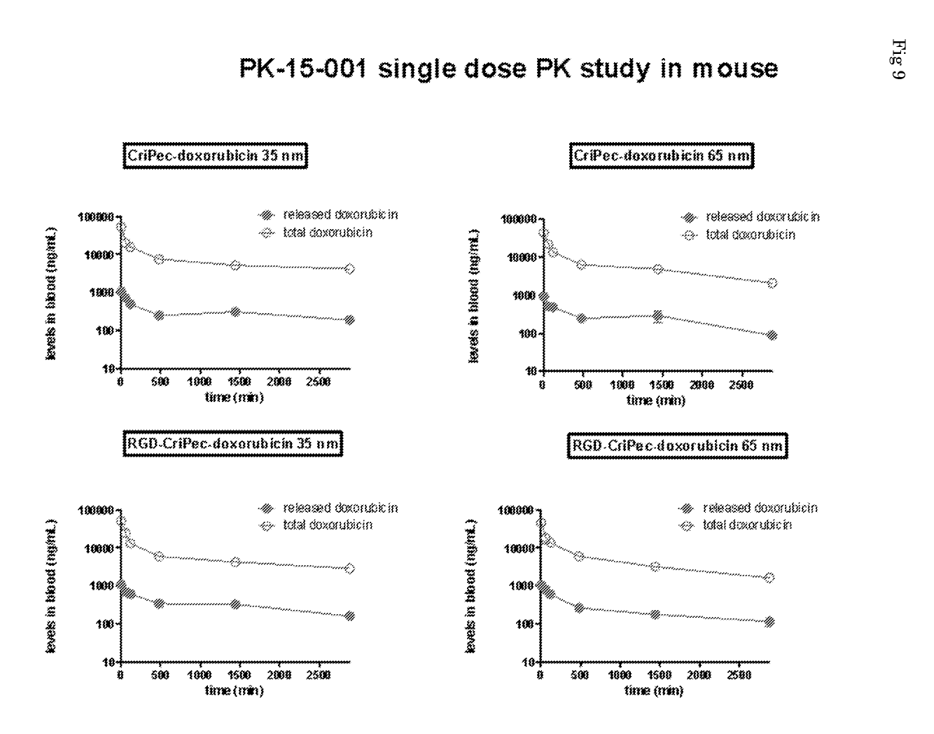

FIG. 9 pharmacokinetic profile for total and released doxorubicin for 35 nm nanoparticle with entrapped doxorubicin without RGD (left upper corner) and with 1% RGD (left bottom corner) and 65 nm nanoparticle with entrapped doxorubicin without RGD (right upper corner) and with 1% RGD (right bottomcorner)

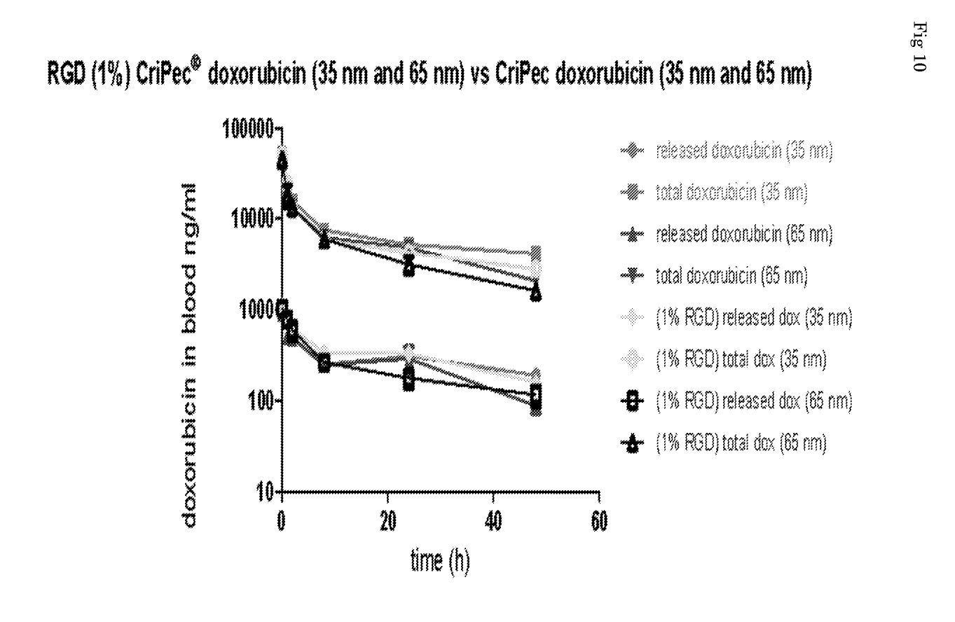

FIG. 10 combines the released and total doxorubicin measured and shows that there is no difference for the different sizes and whether the particles are conjugated with RGD (1%) or not.

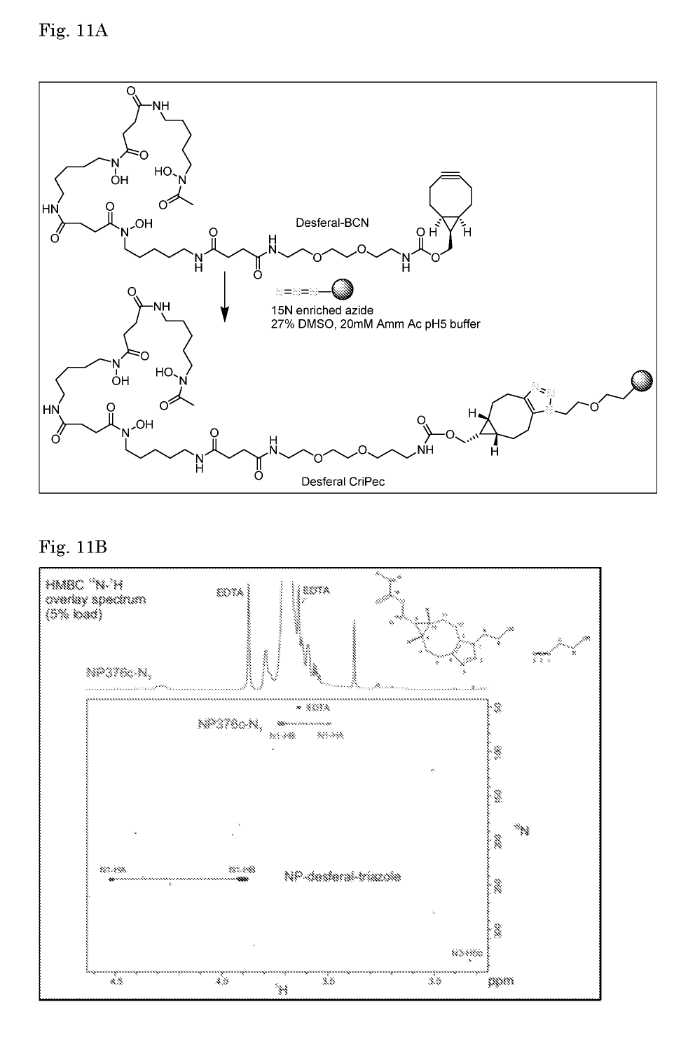

FIG. 11: A. Reaction of desferal-BCN with N₃-CriPec nanoparticle. B. NMR overlay spectrum of the reaction between N₃ CriPec nanoparticles and desferal-BCN.

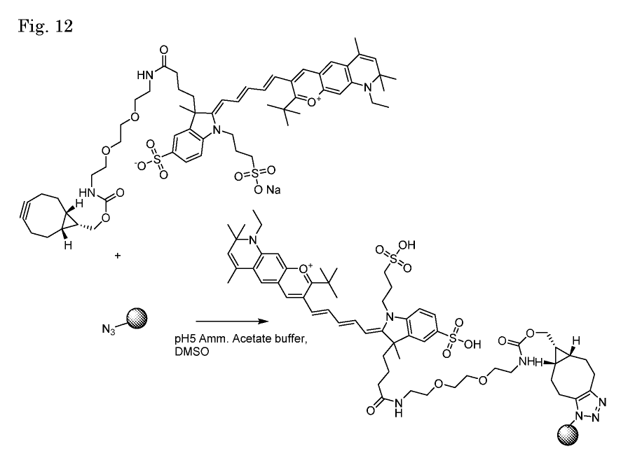

FIG. 12: Reaction of BCN-DY751 with N₃-CriPec nanoparticle.

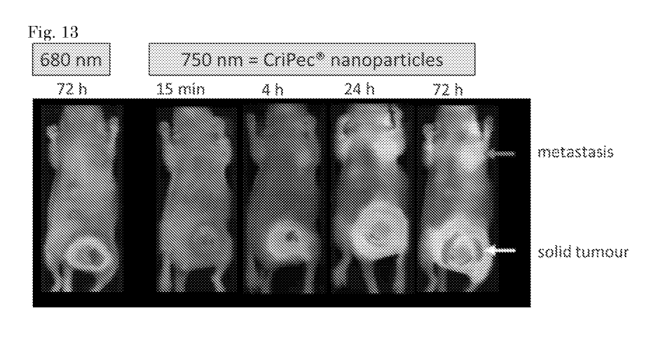

FIG. 13: Accumulation of CriPec® nanoparticle in primary tumour and metastases in nude mice injected with 4T1 breast cancer cells.

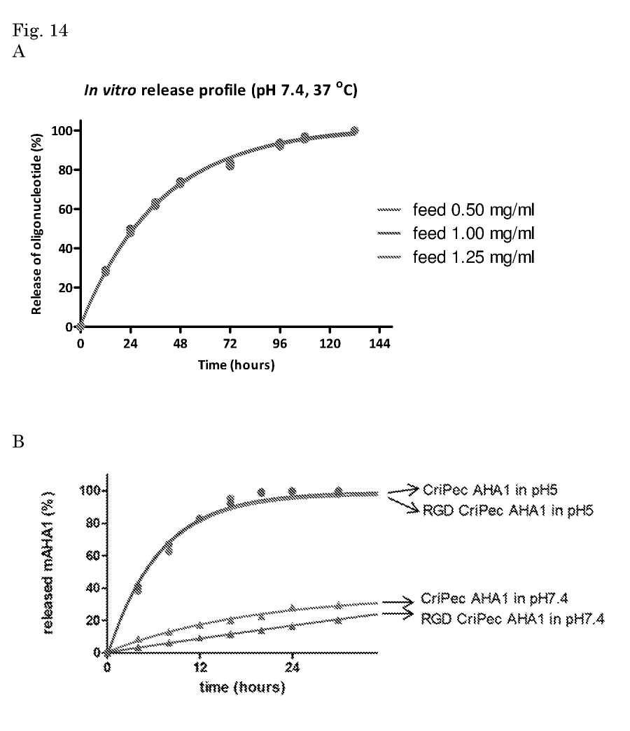

FIG. 14. A. AHA1 release from nanoparticles with and without RGD targeting ligand under physiological conditions (pH 7.4). B. AHA1 release from nanoparticles with and without RGD targeting ligand under slightly acidic (pH 5.5) and physiolocial conditions (pH 7.4).

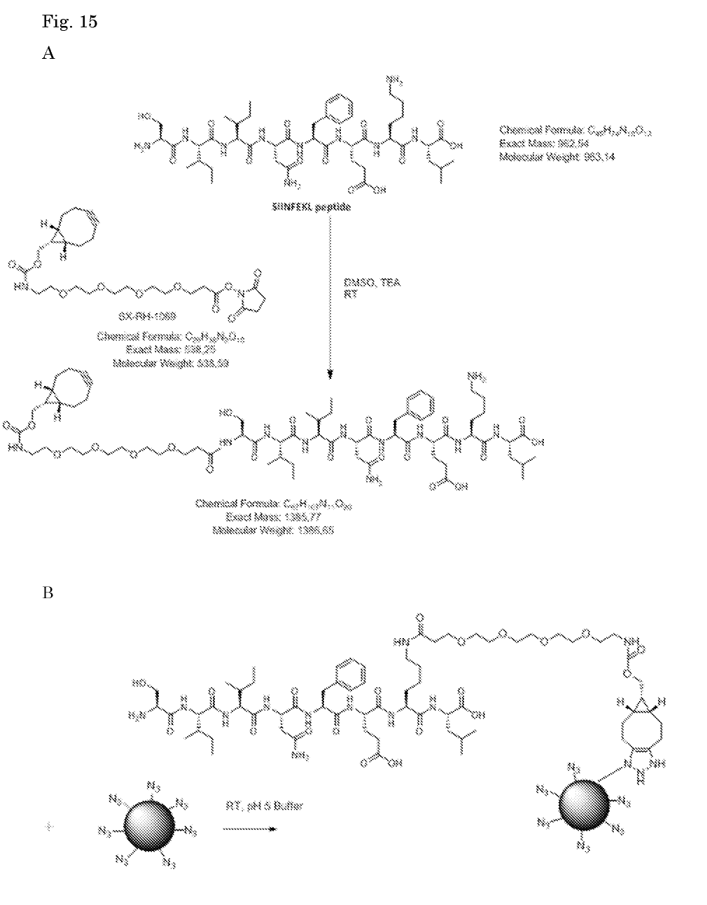

FIG. 15. A. Conjugation of BCN-PEG4-NHS to the terminal NH₂ of SIINFEKL. B. Conjugation of SIINFEKL-BCN to 5% N3 CriPec empty.

The present invention provides for a particle wherein the particle comprises a drug. The particle may covalently entrap a drug on the inside of the particle. In addition, it may also provide for a ligand or drug on the outer surface of the particle. The controlled release particle of the present invention may simultaneous carry several different drugs in one particle, thereby ensuring that the different drugs are released at the same site. As the particles of the invention have a high loading capacity, more than one different drug may be entrapped in the particle. The high loading capacity of the particle also enables the use of less active drugs. It may also be possible to target the controlled release particle to a specific target site, for example by conjugation of a specific ligand to the outer surface of the cross-linked particle. In addition, the release profile of the drug may be tuned as desired. The particle of the present invention may use different linkers for different molecules and drugs, thereby providing the desired release for each drug. One system may be produced with different molecules, each having its own release profile. The system of the present invention provides a true tuneable system for optimisation of the (therapeutic) effect.

The present invention further provides for a particle comprising one or more ligands covalently attached to the surface thereof and method for preparing such particles. Examples of ligands that can be attached to the surface are targeting ligands, therapeutic ligands, imaging ligands or combinations thereof. Such particles may or may not comprise an entrapped drug.

Particles comprising an imaging ligand are particularly useful for (non-) invasive imaging, both in vitro or in vivo. Such particles advantageously further comprise a targeting ligand so that the particles are targeted to a specific part of a compound, system, cell or tissue for imaging. Particles comprising an imaging label are preferably used for imaging and/or as a diagnostic. For instance, such particles can be used as a companion diagnostic. The term “companion diagnostic” as used herein refers to a diagnostic particle used as a companion to a therapeutic compound, such as a drug entrapped particle according to the invention, e.g. to guide treatment decisions for a specific patient. A companion diagnostic is for instance administered to a patient prior to therapy with drug entrapped particles, e.g. to determine the distribution of the particles and thus their applicability and required dosage for a specific patient. If the same nanoparticles (e.g. same size and polymer chains) are use in the diagnostic particles as for the therapeutic particles, the diagnostic particles are particularly suitable to provide insight into the pharmacokinetic and biodistribution profile of therapeutic nanoparticles prior to therapy. This also allows for assessment of differences in the different profiles of nanoparticles with variable pharmaceutical features as size, degradation profile etc, to enable selection of the most suitable therapeutic particles prior to therapy. Particles according to the invention comprising both an covalently entrapped drug and an imaging ligand attached to their surface or a therapeutic ligand and an imaging ligand attached to their surface are for instance used for combined imaging and therapy, i.e. as a theragnostic.

The particle of the present invention is flexible as it comprises the essential required elements for tailor-made optimised drug entrapped particles particle in an optimal manner by full control over:
Possibility of a range of reactive moieties at the outer surface of the particles that allow for the covalent conjugation of one or more specific molecule(s), such as a ligand.
The method of making the particle provides particles with a specific size with a very small particle size distribution, which allows for clear evaluation of specific effects of the particle and prevents unwanted disturbance side effects by a small percentage of very large or very small particles as present in more heterogeneous nanoparticle dispersion such as disclosed in the prior art. From the prior art is appears that for each purpose the particle needs optimisation. The present system, due to its homogeneity, stability, and purity, offers the advantage that one can truly optimise the particle. Particles may be made with no impurities such as much larger or smaller particle, or free drug or ligand. As the drug is covalently entrapped the particles may be easily purified from free drug. The particles are also stable over time during storage as the drugs are covalently linked to the polymer matrix. In this way one is sure that the observed effect is from the particle intended and not from an impurity. In addition, the conjugation of the ligand to the particle is very mild and thus has no significant effect on the drug entrapped in the particle. Furthermore, conjugation of the ligand to the surface is very efficient, generates a stable bond and results in that very little or even essentially no ligand is removed upon treatment such as purification and formulation. Importantly, the exact amount conjugated can be monitored, and in this way one is sure that the observed effect is from the ligand targeted particle intended and not from an impurity as a free ligand or similar. Also the conjugation of a ligand has a minimal effect on the size of the nanoparticle. This enables the use of the particle with different ligands without the need for additional optimisation.
The present method to produce the particles provides the flexibility to produce particles with several different combination of more than one different drug being present inside the polymer matrix as well as ligands on the surface of the particles formed. After crosslinking and/or surface modification, the particle with drug is truly one single macromolecule that allows for ease of purification. This high purity is not only essential to evaluate the underlying mechanism of action with regard to therapeutic action in detail, but also represents a major advancement in terms of drug safety and pharmaceutical characterisation.
The present invention allows for entrapment of a range of compounds, either hydrophilic as well as hydrophobic, and over a large size range. The present inventive particles are not restricted to hydrophilic or hydrophobic active agents. The cross-linking of the drug to the polymer matrix allows both hydrophobic and hydrophilic adjuvants and drugs to be entrapped into the polymer matrix.
The present invention allows for the covalent entrapment of a wide variety of drugs of various classes, such as a macromolecule, protein, peptide, hormone, small molecule, e.g. synthetic chemical entity, or nucleic acid molecule such as mRNA, siRNA, shRNA and DNA molecules, aptamers, or any combination thereof.
The drug may be a protein or peptide and may be susceptible to chemical and enzymatic degradation as well as physical alteration like aggregation or precipitation upon exposure to physiological conditions. The particle of the present invention may provide entrapment of proteins thereby protecting them against (enzymatic) degradation after introduction to the body. The particles may be very dense, thereby inhibiting penetration of enzymes to the core of the particle, and thus effectively protecting the proteinaceous drugs.
The particle of the invention may be compact and intact, thus limiting macrophage uptake, so more therapeutic targeting is possible. The particles of the invention have shown a long blood residence as well as high accumulation in tumour and in inflamed tissue.
The particles of the invention are initially stable due to the crosslinking, but are also in time biodegradable. The stability prevents a burst release, and keeps the drug longer in the circulation, thereby increasing the possibility to activate the right target cells over a longer term period. In time, the entrapped compounds, such as drug, are being released. In addition, the particles of the present invention disintegrate into small fragments.
The particle of the invention may be tuned to a desired drug release kinetics. The type of crosslinking and the cross-link density may be tuned to obtain a desired degradation rate. Below experiments show that cross-link type and cross-link density determine the kinetics of the drug release. In this way the particle is tuned for a desired drug release kinetic, from short to long. For example the degradation of the particle may take about 30 days under physiological conditions or 200 days or even about 400 days depending on the type of linker and the cross-link density.
In the particle of the invention it is possible to tune the drug linker type and thus particle with long(er) lasting drug exposure may be made. It is possible to conjugate the drug with different linkers.

Different release profiles for drugs are then possible. Drugs may have different reactive groups, however the system allows different linkers and thus these different linkers may be used to link different reactive groups on the drugs thereby allowing more different drugs to be used. This provides more control and linker specificity for derivative formation and/or purification and allows greater flexibility. The different linkers also allow different release rates for drugs from one particle, for example for use of different drugs or one kind of drug but then both a fast and a slow release.
The method of the present invention is very flexible. It provides the synthesis of particles, having covalent drug entrapment and optionally additionally covalent conjugation of ligands to the surface.
The method of the present invention allows easy purification to remove any non-covalently entrapped drug or ligand as the drug is covalently entrapped and thus stabile in the particles of the invention as well as the ligand stably conjugated to the surface. The particle of the invention is very controllable and also broadly applicable, with high batch to batch reproducibility that allow for clear evaluation of 1 parameter at a time. This will ease the optimisation of the production as well as the optimisation of the therapeutic use.

Particularly, in one embodiment, the present invention results in drugs, entrapped, optionally covalently in or coupled to polymer carriers or polymeric devices, such as micelles, nanoparticles, microspheres, hydrogels and other types of polymer carriers or devices for vaccination; the drugs are covalently entrapped within the particle and/or bonded to the polymeric devices or carriers.

In a embodiment of the invention and/or embodiments thereof, the particle is a controlled release system, and may encompass all kinds of controlled release, including slow release, sustained, pulsatile and delayed release.

For the present invention it should be understood that the particle may be a nanoparticle and/or a microparticle.

Nanoparticles are considered to be promising candidates for therapeutic use against diseases. The particle of the present invention may contain a broad variety of drugs including both hydrophobic and hydrophilic compounds. A suitable particle is described in WO 2010/033022.

In the particle of the present invention and/or embodiment thereof drugs are first non-covalently entrapped in polymer phases, and especially in polymer-rich phases, in an aqueous environment, and subsequently are covalently conjugated to a 3D-polymer network.

In step (ii) of the methods to prepare drug entrapped particles, formation of the particles, the drug and/or drugs are physically, or non-covalently entrapped. Or in the alternative method, in step (iii) wherein the drug are mixed with the formed particle, the drug are physically, or non-covalently entrapped. In the crosslinking step, the drug and/or drugs are covalently entrapped, rendering a particle wherein the drug is covalently entrapped in the inside of the particle. It should be noted that the prior art discloses systems wherein first a cross-linking step is performed without the present of the drug. In the invention of the application, the drug are present during the cross-linking step thereby covalently linking the drug to the polymer matrix. Also when linking the drug to the surface of the particle, the drug is covalently linked to the polymer matrix of the particle.

The particle of the present invention and/or embodiments thereof are prepared by first mixing a drug with a polymer and then subsequently cross-linking the polymer to form a polymer matrix. The crosslinking may be done with polymer and drug each derivatised with polymerisable moieties and in the presence of free-radical initiators, but also other types of covalent conjugation linker are possible.

Particles with covalently entrapped and/or conjugated drugs may have several advantages as explained above. The resulting particles may have therapeutic, curative or prophylactic properties. The particle of the present invention and/or embodiments thereof may provide a tuneable system for providing the drug to the location where it is needed. In addition, the particle may be decorated with ligands, to target to a desired location and/or particular cell type. Entrapment of drugs in a particle or by conjugation of a therapeutic ligand may make these compounds suitable for treatment, e.g. by oral or subcutaneous administration.

In step (i) the polymer chains optionally interact with each other (see herein-below) forming polymer sub phases in an aqueous phase. That is, relatively, polymer chain-rich and relatively polymer chain-poor phases are created. In a preferred embodiment, the drug is present in the polymer chain rich phases. A sub-location of drug in polymer chain rich sub-phases occurs based on physical interactions between the drug and the polymer chains.

In step (i), the drug do not form covalent conjugates with the polymer chains. Only in the cross-linking step (ii) or (iii) the drug and the polymer chains together form a 3D-network.

The drug are covalently bonded to the polymer carrier, optionally via linker molecule, simultaneously with the cross-linking of the polymers forming the polymeric carrier or device. The cross-linked drug-polymer conjugates which are formed in step (ii) or (iii) exhibit a higher thermodynamic stability than the non-cross-linked polymer particles. In addition, the entrapped drug molecules are prevented from rapid release due to covalent bonding to the polymeric carrier.

The particle of the invention does not require the coupling of the drug directly to single polymer chains up-front to particle formation, thereby fully retaining the initial properties of the polymers used, such as thermo-sensitive properties and/or the ease of drug loaded particle formation. The use of a fixed type of polymer, for example thermo-sensitive biodegradable block copolymers, provides a broadly applicable platform technology that allows a rapid and easy change/optimization of the composition of the drug entrapped devices.

The particle of the present invention is applicable to all drugs that are capable of non-covalently interacting with polymer chains which are capable of forming polymeric carriers after cross-linking. In the aqueous phase, the polymer chains (before the cross-linking step) optionally assemble in a certain structure, or at least in polymer chain-rich domains; and the drug localise in these assemblies. All types of physical interactions are possible (see below).

The only further requirement is that the drug contains a moiety (or can be modified with a reactive substituent) that is capable to react with a moiety of the polymer chains that form the basis of the polymeric particle. Optionally the drug does not comprise an alkyne group or an azide group.

In a preferred embodiment, the drug is provided with a linker molecule, optionally a degradable linker. Hence, in a preferred drug entrapped particle of the invention, the drug is attached to the polymer matrix via a degradable linker.

By covalent entrapment of the drug in the core of the carrier, such as in the particle core, the drug does not come free at the beginning, it does not have a “burst release”. It will benefit from the prolonged residence and/or blood circulation of the cross-linked carrier in the body, thereby acting as a depot on the injection site and/or in the blood stream while simultaneously, this can lead to elevated drug concentrations in the target tissue e.g. tumour, lymph node, or inflamed tissue. In addition, the particle of the present invention may obtain a long term product stability by subjecting these to lyophilisation. For example, particles according to the present invention comprising drug-loaded particles may easily be freeze-dried and subsequently suspended without loss of morphology; as dry powder, a long shelf life is obtained. This is advantageous as especially in developing countries, particles that do not need refrigeration, and/or are a dry powder are preferred.

The resulting drug-loaded polymeric devices, do not display a premature release of drugs (burst release), but demonstrate a prolonged residence at site of injection and/or blood circulation e.g. upon parenteral administration. In a embodiment of the invention and/or embodiments thereof, the drug comprises a suitable linker that allows sustained release of entrapped compounds in time, optionally each with its own specific release rate. This may result for instance in a (greatly) enhanced cancer cell targeting, and accumulation in the cancerous tissue, thereby increasing the therapeutic action.

In a embodiment of the invention and/or embodiments thereof, the drug is entrapped via linker to the polymer matrix, optionally a degradable linker or optionally a biodegradable linker. Such a system allows a pulsatile or constant release of the drug. Controlled release of the drug from the carrier is accomplished by cleavage of the, optionally degradable, linker or linking group between the drug, and the polymeric carrier under physiological conditions, or by local environmental triggers or external stimuli as explained and elaborated, herein-below. In addition, the entrapment prevents exposure of blood to toxic high drug peak levels that would otherwise be present immediately after intravenous administrations of free drugs, or in non-covalently entrapped drugs. More importantly, by preventing migration of the system to normal tissues, acute toxic effects may be diminished. The other way around, the drug are fully protected from the environment by confinement in the formed three-dimensional network of the cross-linked polymer carrier, such as a cross-linked micellar core, thereby preventing a premature degradation and/or clearance. These unique aspects deliver the drug at the right place and time, and at an anticipated efficacious dose.

Alternatively, in a embodiment of the invention and/or embodiments thereof, ligands on the surface of the particles may not need to be released, as they will be available for the cells to be targeted by being present on the outer surface of the particles.

The stepwise method of making the particle of the particle of the invention comprises two essential consecutive steps.

In the first step, a cross-linkable polymer and a drug are mixed in an aqueous environment. This is optionally achieved by adding the drug, optionally in a suitable solvent may be water or a water miscible solvent such as a lower alcohol like ethanol, tetrahydrofuran, or dimethylsulphoxide to an aqueous polymer solution or dispersion. The polymer present and the drug are selected so that the polymer and the drug will be in intimate contact, and in a preferred embodiment, the drug is in contact with the polymer chains. Said in other words, in the first step physical, non-covalent interactions between the polymer chains and the drug result in the selective localisation of compounds in specific regions of a polymeric device.

As a result of the first step, the molecules forming the drug are non-covalently entrapped in and between the polymer chains in solution. In the present description and the appending claims, the concept of “non-covalent interaction” means any interaction which is not covalent, i.e. any bonding between atoms or bonds which bonding does not involve the sharing of electron pairs. Examples of non-covalent interaction are hydrophobic, aromatic, hydrogen bonding, electrostatic, stereocomplex, and metal-ion interactions.

In the cross-linking step of the method of making the particle of the particle of the invention, following the first step, the non-covalently entrapped drugs are covalently coupled to the newly forming/formed polymer network. That is, a reaction is carried out, wherein the polymer chains are cross-linked. This can occur both inter- and intramolecularly, but the intermolecular cross-links are clearly preferred and any steps that favour intermolecular cross-linking are preferred embodiments of the presently claimed process. Simultaneously with the cross-linking step, the reactive moieties of the drug are also co-crosslinked to the polymer chains and an intertwined network of the polymers and drug is formed. Suitably, the polymer comprises more than one reactive group and may react with more than one drug. Optionally, the polymer comprises different reactive groups that are capable of reacting with each other, thereby forming a 3-D network of the polymer and the drug. Polymers that comprise two or more different reactive groups may be used. In addition, the different reactive groups may be present on different polymers. Optionally, the reactive groups are not an azide group. Optionally, the reactive groups are not an alkyne group.

This step may require initiators and/or catalysts, but also physical circumstances may lead to the reactions forming cross-links and conjugates. In case initiators and/or catalysts are required, these may be added to the polymer solution together with the drugs, but can also be added to the reaction system at an earlier or later stage

Since the degree of incorporation of drug, the entrapment efficiency, may be as high as 95-100%, a high amount of drugs may be incorporated in the formed 3D-network.

The loading capacity of the particles of the invention may be at least 10%, 11%, 12%, 13%, or 14% or even at least 15%, 16%, 17%, 18%, or 19% or even at least 20%, 21%, 22%, 23%, or 24%, or even at least 25%, 26%, 27%, 28%, or 29% and even at least 30%, at least 32%, alt least 35%, at least 37% or even at least 40%. Preferably the loading capacity of the particles of the invention is at least 10%, more preferably at least 12%, more preferably at least 15%, more preferably at least 17%, more preferably at least 18%, more preferably at least 20%, more preferably at least 21%.

The loading capacity is the amount of drugs entrapped by weight divided by the weight of the particle or polymer expressed in %. Increasing the drug feed or drug concentration with respect to the polymer feed or polymer concentration in the preparation of the particles, either in step (i) or step (ii) increases the loading capacity. Typical drug concentration or drug feed may range from 0.1 mg/ml to 50 mg/ml. Optionally the drug feed or drug concentration ranges from 0.5 mg/ml to 40 mg/ml, optionally from 1 mg/ml to 30 mg/ml, optionally from 1.5 mg/ml to 25 mg/ml, optionally from 2 mg/ml to 20 mg/ml, optionally from 2.5 mg/ml to 15 mg/ml, optionally from 3 mg/ml to 12 mg/ml, optionally from 3.5 mg/ml to 10 mg/ml, optionally from 4 mg/ml to 8 mg/ml, optionally from 4.5 to 7 mg/ml, optionally from 5 to 6 mg/ml.

The ratio drug:polymer by weight optionally ranges from 0.01 to 10, optionally from 0.05 to 8, optionally from 0.1 to 6, optionally from 0.15 to 5, optionally from 0.2 to 4, optionally from 0.25 to 3, optionally from 0.3 to 2.5, optionally from 0.35 to 2, optionally from 0.4 to 1.5, optionally from 0.45 to 1, optionally from 0.5 to 0.75.

It was also found that the drug entrapment efficiency of the particles of the present invention is surprisingly high. The drug entrapment efficiency is the weight of the drug entrapped divided by the weight of the drug fed to the particle expressed in %. For the particle of the invention, the drug entrapment efficiency may be at least 30% or even at least 40%, at least 50%, at least 60%, at least 70% and even at least 80%.

It was found that the loading capacity of the present particles does not influence the size distribution. Thus particles with loading capacity of 10%, 11%, 12%, 13%, or 14% or even at least 15%, 16%, 17%, 18%, or 19% or even at least 20%, 21%, 22%, 23%, or 24%, or even at least 25%, 26%, 27%, 28%, or 29% and even at least 30%, at least 32%, alt least 35%, at least 37% or even at least 40% may have a narrow polydispersity index (DPI) of less than 0.5, optionally less than 0.4, even optionally less than 0.3, optionally less than 0.2 and optionally less than 0.1, or optionally less than 0.05.

Thus particles with loading capacity of 10%, 11%, 12%, 13%, or 14% or even at least 15%, 16%, 17%, 18%, or 19% or even at least 20%, 21%, 22%, 23%, or 24%, or even at least 25%, 26%, 27%, 28%, or 29% and even at least 30%, at least 32%, alt least 35%, at least 37% or even at least 40% may range in size between 0.1 and 1000 nanometer, optionally between 1 and 500 nanometer, optionally between 5 and 250 nanometer, optionally between 10 and 200 nanometer, and optionally between 30 and 150 nanometer.

According to a preferred method of the present invention and/or embodiments thereof, amphiphilic polymers may be fully dissolved in a solvent.

Drugs may be present in the solvent or may be added after the dissolution of said polymers or even upon self-assembly into loose particle, and the drugs will form a general distribution over the polymer or micellar solution;

Then, this system may be subjected to a change of certain circumstances (e.g. temperature, pH, solvent) leading to a situation that at least parts of the polymers display a different behaviour than other parts of the polymers and clustering takes place;

due to the physical properties of the drug, these drug localise in certain regions of the newly formed clustered polymeric solution;

after this localisation, cross-linking takes place to fixate the drug in their preferred regions.

Optionally thermosensitive block copolymers are used. For example, the drug is mixed in an aqueous environment, wherein also a non-cross-linked thermosensitive block copolymer is present at a temperature lower than its Lower Critical Solution Temperature (LCST) or lower than its critical micelle formation temperature (CMT). At any temperature below this LCST, the system is in solution; at any temperature below this CMT, micelle formation does not occur. However, by heating such systems, particles or particle are formed thereby entrapping the drug in their hydrophobic core. Alternatively, empty particle are formed in step (i) without the drug. Subsequently a drug solution is added to the empty particle. Next, the cross-linking reaction that forms the intertwined micellar network in the core is also carried out at a temperature higher than the LCST or the CMT. This cross-linking reaction can be accelerated by the addition of an initiator/and or catalyst, either prior to heating of the polymer solution or after formation of the non-cross-linked particles or particle. The method of forming first the nanoparticles and then adding the drug before the crosslinking may be very suitable for peptides.

In another embodiment of the method of the invention and/or embodiments thereof, the polymers do not need harsh conditions for making the particles. Suitably, the formation of the particles is without organic solvents and/or other chemicals or solvents that may harm the drug. Suitable polymer chains that may be used in the present invention are, e.g., thermo-sensitive block copolymers. Particularly, copolymers based on PEG-b-poly(N-hydroxyalkyl methacrylamide-oligolactates) with partially methacrylated oligolactate units are preferred. Various other (meth)acrylamide esters can be used to construct the thermosensitive block, e.g. esters, and optionally (oligo)lactate esters, of HPMAm (hydroxypropyl methacrylamide) or HEMAm (hydroxyethylmethacrylamide), and N-(meth)acryloyl amino acid esters. Preferred thermo-sensitive block copolymers are derived from monomers containing functional groups which may be modified by methacrylate groups, such as HPMAm-lactate polymers.

Other types of functional thermosensitive (co)polymers, which may be used, are hydrophobically modified poly(N-hydroxyalkyl)(meth)acrylamides, copolymer compositions of N-isopropylacrylamide (NIPAAm) with monomers containing reactive functional groups (e.g., acidic acrylamides and other moieties such as N-acryloxysuccinimide) or similar copolymers of poly(alkyl) 2-oxazalines, etc.

Further preferred thermo sensitive groups may be based on NIPAAm and/or alkyl-2-oxaxolines, which monomers may be reacted with monomers containing a reactive functional group such as (meth)acrylamides or (meth)acrylates containing hydroxyl, carboxyl, amine or succinimide groups.

Suitable thermo-sensitive polymers are described in U.S. Pat. No. 7,425,581 and in EP-A-1 776 400.

However, also other types of amphiphilic block copolymers or ionic particle that are not necessarily thermo-sensitive and contain or may be modified with cross-linkable reactive groups, may be used. In such cases state-of-the-art methods can be used to form the particles and/or particle, such as direct dissolution, dialysis, salting-out and solvent-evaporation.

These other types of polymers that conform polymer-rich phases in water (e.g. due to hydrophobic interactions or ionic interactions) and that contain reactive moieties or contain moieties that can be used to couple reactive moieties, e.g. PEG-PLA-methacrylate (e.g. as described in detail in Kim et al., Polym. Adv. Technol., 10 (1999), 647-654), methacrylated PLA-PEG-PLA (e.g. as described by Lee et al. in Macromol. Biosci. 6 (2006) 846-854), methacrylated PEG-poly caprolactone (e.g. as described by Hu et al. in Macromol. Biosci. 9 (2009), 456-463), as well as other reactive moieties containing (block co)polymers based on poly lactic acid, poly lactic acid glycolic acid, and/or poly caprolactones.

In addition, polymers capable of forming a particle because of ionic interactions may be used, such as block ionomer complexes of poly(ethylene oxide)-b-polymethacrylic acid copolymers and divalent metal cations (e.g. as described by Kim et al. in J. Control. Rel. 138 (2009) 197-204, and by Bontha et al. in J. Control. Rel. 114 (2006) 163-174) polyionic complexes based on block copolymers of poly(ethylene glycol) and poly(amino acid) (e.g. as taught in Lee et al., Angew. Chem 121 (2009) 5413-4516; in Nishiyama et al. in Cancer Res. 63 (2003), 8977-8983, or in Miyata et al., J. Control. Rel. 109 (2005) 15-23.

In general, all polymers that are able to create different subphases in a suitable solvent system can be used, together with a drug that can localize selectively in such subphases.

The polymer chains and the drug contain or may be modified such that these contain reactive moieties. The polymers used should contain a sufficiently high number of reactive substituents capable of cross-linking and reacting with the reactive groups of the drug. Suitable results are obtained when for instance 10-15%, 15-20%, 20-25%, 25-30%, 30-35%, 35-40%, 40-45%, or 45-50% of the monomer units of the polymer have a reactive substituent; however also up to 100% of the monomer units may be derivatised with reactive substituents. For example 50%, 55%, 60%, 65%, 70%, 75%, 80%, 85%, 90% or 95% of the monomer units may be derivatised with reactive substituents. Also 1-10%, 2-8%, 3-7%, 4-6%, and 2-5% of the monomer units may be derivatised with reactive substituents.

Also the drug have reactive substituents that are capable of crosslinking, optionally to the polymers so that an drug-polymer matrix is formed. In a preferred embodiment of the present invention and/or embodiments thereof, the drug have at least one reactive moiety or substituent that is capable of cross-linking. Optionally, more than 1, such as 2, 3, 4, or 5 reactive moieties are present on the drug. Optionally the drug does not comprise an alkyne group. It should be understood that larger molecules may have more reactive moieties than smaller molecule, and it thus the amount of reactive moieties largely depends on the size of the drug. Drugs may be large biomolecules and hence may contain more than 5, or even more than 10, or even more than 15, or even more than 20 or even more than 25 reactive moieties. In the context of the present invention, reactive moiety, reactive substituent, and reactive group are used interchangeably and all mean a group that is capable of an action such as cross-linking and linking to another molecule. Optionally, the reactive group or reactive moiety is not an azide group. Optionally, the reactive group or reactive moiety is not an alkyne group.

The release rate of the drug can easily be controlled by using different type of linkers to conjugate the reactive moiety to the drugs. Suitable types of well-known degradable linker molecules include but are not limited to esters, carbonates, imines, carbamates, succinate or ortho (oxime) esters, ketals, acetals, hydrazone, and enzymatically degradable linkers (e.g. peptides) or a combination of these. In addition, all kinds of well-known stimuli sensitive linkers, such as photo-/temperature-/ultrasound-sensitive and other linkers can also be used. When modifying drugs, one takes care of the type of conjugation such that upon release, only the drug is released and no derivatives that may have other activities, as to assure its full activity. By using a biodegradable linkage, the original drug, will be released according to a specific controlled release profile and subsequently exert its activity and especially its immunogenic or stimulating effect.

Particle of the present invention are polymer carriers, such as micelles, nanoparticles, microspheres, hydrogels and other types of polymer carriers or devices comprising entrapped or otherwise incorporated drugs for controlled release, such as devices with a coating with entrapped drugs.

As said, in crosslinking step is essential for the method of the invention. Suitable crosslinking according to the invention is cross linking resulting in a bond selected from the group consisting of ester, hydrazine, amide, Schiff-base, imine, acetal bonds, and similar biodegradable bonds, including any potential corresponding derivatives of them. Suitable crosslinking according to the invention is cross linking with a reactive moiety selected from alcohol, acid, carboxyl, hydroxyl, amine, hydrazine, etc. Also photopolymerisation is suitable (Censi et al J. Control Rel 140 (2009) 230-236). The reactive moieties may be present in the polymer chain, and/or in the drug and/or on a linking molecule. Optionally the linker or polymer comprises more than 1 reactive moiety so as to form multiple bond.

Optionally the crosslinking results in biodegradable linkages.

When the drug is entrapped via degradable linker, a constant release of the therapeutically active compound is assured. Controlled release of the drug from the carrier is accomplished by cleavage of the, optionally degradable, linker or linking group between the active ingredient, such as drug, and the polymeric carrier under physiological conditions, or by local environmental triggers or external stimuli as explained and elaborated, herein-below. A suitable example of degradable linker may be found in WO2012/039602 which is incorporated by reference.

Such a linker can be exemplified by the following formula:

HOQ-(CₙH₂ₙ)—S(R₁)(R₂)—(CₘH₂ₘ)—CH₂-A,

wherein n and m are integers from 0 to 20, and optionally from 1 to 10. Optionally n is an integer from 1-5, more optionally from 1-3; and m is an integer from 1-7; more optionally from 1-5;
wherein R₁ and R₂ are independently from each other selected from an electron lone pair, an oxygen moiety, such as ═O, a nitrogen moiety, such as ═N—Rₓ, wherein Rₓ is a homo- or heterogeneous group of atoms, and optionally, independently, a straight or branched C₁-C₆ alkyl, a straight or branched C₁-C₆ alkenyl, which alkyl or alkenyl group may optionally be substituted by one or more halogen groups, hydroxyl groups, amino or substituted amino groups, carboxylic acid groups, nitro groups or cyano groups; or aromatic groups, and optionally a phenyl group optionally be substituted by one or more of the substituents mentioned for the alkyl and alkenyl groups; or a halogen group, a hydroxyl group, an amino group, or a substituted amino group (the substituents being one or two C₁-C₃ alkyl groups), a carboxylic acid group, a nitro group, or a cyano group;
wherein A is a conjunction moiety; and
wherein Q is a direct bond, a C═O, a C═NH or C═NRₚ group, wherein Rₚ is a C₁-C₃ alkyl. In this formula the HO-Q group can be replaced by a HR₉N-Q group, wherein R₉ can either be a hydrogen atom or a C₁-C₃ alkyl group.

In the following preferred linker formula, the HO-Q group is a carboxylic acid group and the conjugation moiety A is a polymerisable methacrylate, which moieties are also exemplified in the working examples herein-below:

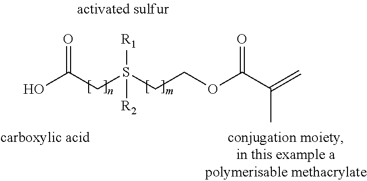

It should be understood that the above example is not limited and that for example the methacrylate group may be substituted with any polymerisable group as described in the specification. Suitable conjugation groups are polymerisable moieties of the formula —PL-RᵥC═CRᵤR_{w}, wherein —PL- is a linking group such as an —O—, a —NH—, a substituted —N—, the substituent being a C₁-C₃ alkyl, an —O—C(O)—, an —O—(C(O))ᵣ—C₆H₂₆—, wherein r is 0 or 1, and b is an integer from 1 to 6; and Rᵤ, Rᵥ and R_{w}, independently, represent a hydrogen atom or a C₁-C₃ group.

Optionally the end terminal of the polymer comprises an azide group or alkyne group that may interact with a ligand that comprises an alkyne group or azide group respectively. The azide group or alkyne group only at the end terminal of the polymer, ensures that the physical-chemical properties of the polymer are unchanged, while an additional functionality is employed. It was surprisingly found that the azide group or alkyne group may undergo particle formation and cross-linking reactions and remains active after these steps for formation of a bond to a alkyne group or azide group on a ligand. Suitably azide group or alkyne may be introduced by starting from respectively an azide-PEG-OH molecule or an alkyne-PEG-OH molecule and convert this in an initiator instead of the mPEG-OH. The azide-PEG initiator or alkyne-PEG initiator is then used to derivatise the polymer chain with a azide group or alkyne group respectively. In the present invention, optionally between 1 and 30 wt. %, more optionally between 2 and 15 wt. %, and most optionally between 3 and 10 wt. %, such as up to 6 wt. % of the copolymer chains may contain azide groups.

In any aspect of the invention and/or embodiment thereof the polymer chain comprises at least one azide group per polymer chain, optionally one azide group per polymer chain, optionally at the end position.

In any aspect of the invention and/or embodiment thereof the polymer chain comprises at least one alkyne group per polymer chain, optionally one alkyne group per polymer chain, optionally at the end position.

The present invention thus is directed to methods and products wherein the polymer is functionalised, or derivatised by an azide group and the ligand is functionalised or derivatised by an alkyne group.

The present invention thus is directed to methods and products wherein the polymer is functionalised, or derivatised by an alkyn group and the ligand is functionalised or derivatised by an azide group.

In any aspect of the invention and/or embodiment thereof, 1 out of 1500 polymer chains are derivatised by an azide group, optionally 2 out of 1500, optionally 5 out of 1500, optionally 7 out of 1500, optionally 10 out of 1500, optionally 15 out of 1500, optionally 20 out of 1500, optionally 25 out of 1500, optionally 30 out of 1500, optionally 35 out of 1500, optionally 40 out of 1500, optionally 45 out of 1500, optionally 50 out of 1500, optionally 55 out of 1500, optionally 60 out of 1500, optionally 65 out of 1500, optionally 70 out of 1500, optionally 75 out of 1500, optionally 100 out of 1500, optionally 150 out of 1500, optionally 200 out of 1500, optionally 250 out of 1500, optionally 300 out of 1500, optionally 400 out of 1500 optionally 500 out of 1500, optionally 600 out of 1500, optionally 750 out of 1500, optionally 900 out of 1500, optionally 1000 out of 1500, optionally 1200 out of 1500, optionally 1300 out of 1500, optionally 1400 out of 1500 polymer chains are derivatised by an azide group. It is to be understood that the azide group and polymer chain amount as described here is a ratio and is not to be construed to limit the methods and/or particles by 1500 polymer chains.

In any aspect of the invention and/or embodiment thereof about 0.01% to about 100% of the polymer chains is derivatised by an azide group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the polymer chains is derivatised by an azide group.

In any aspect of the invention and/or embodiment thereof about 0.01% to about 100% of the ligand is derivatised by an azide group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the ligand is derivatised by an azide group.

In any aspect of the invention and/or embodiment thereof 1 out of 1500 polymer chains are derivatised by an alkyne group, optionally 2 out of 1500, optionally 5 out of 1500, optionally 7 out of 1500, optionally 10 out of 1500, optionally 15 out of 1500, optionally 20 out of 1500, optionally 25 out of 1500, optionally 30 out of 1500, optionally 35 out of 1500, optionally 40 out of 1500, optionally 45 out of 1500, optionally 50 out of 1500, optionally 55 out of 1500, optionally 60 out of 1500, optionally 65 out of 1500, optionally 70 out of 1500, optionally 75 out of 1500, optionally 100 out of 1500, optionally 150 out of 1500, optionally 200 out of 1500, optionally 250 out of 1500, optionally 300 out of 1500, optionally 400 out of 1500 optionally 500 out of 1500, optionally 600 out of 1500, optionally 750 out of 1500, optionally 900 out of 1500, optionally 1000 out of 1500, optionally 1200 out of 1500, optionally 1300 out of 1500, optionally 1400 out of 1500 polymer chains are derivatised by an alkyne group.

It is to be understood that the alkyne group and polymer chain amount as described here is a ratio and is not to be construed to limit the methods and/or particles by 1500 polymer chains.

In any aspect of the invention and/or embodiment thereof about 0.01% to about 100% of the polymer chains is derivatised by an alkyne group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the polymer chains is derivatised by an alkyne group.

In any aspect of the invention and/or embodiment thereof about 0.01% to about 100% of the ligand is derivatised by an alkyne group, optionally about 0.1% to about 90%, optionally about 0.5% to about 80%, optionally about 1% to about 70%, optionally about 2% to about 60%, optionally about 3% to about 50%, optionally about 4% to about 40%, optionally about 5% to about 30%, optionally about 6% to about 25%, optionally about 7% to about 20%, optionally about 8% to about 15%, optionally about 9% to about 13%, optionally about 10% to about 12% of the ligand is derivatised by an alkyne group. For particles wherein the surface is not further modified, the polymer may have a non/reactive moiety, such a methoxy, on the end terminal. A suitable polymer is m-PEG-b-HPMAmLacX, whereby X can be any reactive moiety that can interact with an (Y-derivatised) compound, resulting in a stable or biodegradable bond. The latter is dependent on the type of compound use, i.e. when its biological activity is limited by a stable conjugation, it might require a biodegradable bond to assure release in time, allowing for maximum biological effect, whereas for internalizing ligands, a stable bond is required to assure integer intracellular uptake of the entire particle.

Optionally the percentage of the X-polymer may range between 0 and 100%, optionally between 0-50%, optionally 0-20%, and is a good control for the % of reactive moieties that may be conjugated to the surface. Optionally X is azide or alkyne.

In this way, the particles not only entrap drugs but may also comprise ligands on the surface to really target the particles with a drug to the target cells that are required for therapeutic action. Optionally the ligand on the surface of the particle is utilised with a linker, optionally a degradable linker. Suitably the linker comprises a alkyne group or an azide group

Optionally ligands are conjugated to the surface of the particles. The ligand may suitably target drug to specific cells or tissued, such as cancer cells. Any type of compound that is able to target to a specific part of a system to be treated can be used as a ligand. Such ligand is herein also referred to as a targeting ligand. Suitable ligands may be proteins such as antibodies, nanobodies, antibody fragments, growth factors, and transferrin, peptides such as RGD, cyclic RGD, octreotide, AP peptide and tLyp-1 peptide, aptamers such as A10 and AS1411, polysaccharides such as hyaluronic acid, small biomolecules such as folic acid, galactose, bisphosphonates, biotin and small molecules such as synthetic chemical entities. In addition, suitable ligands may be selected from the group consisting of mannose/mannan, ligands for the Fc receptors for each immunoglobulin class, CD11c/CD18 and DEC 205 receptor targets, DC-SIGN receptor targets. A skilled person is well aware of suitable targeting compound(s)/ligands for desired target cells and is able to select the desired targeting compounds. In the context of this invention, targeting ligand, targeting, targeting compound, targeting group or ligand are used interchangeably, and all mean a compound that is able to target a specific cell or specific tissue. Preferred targeting ligands are peptides and proteins, including antibodies, nanobodies, antibody fragments and growth factors.

Alternatively, the ligand may be or comprise an imaging agent, enabling (non-)invasive imaging. As used herein the term “imaging ligand” refers to a moiety which allows detection of the particles of the invention, e.g. when present in or bound to a compound, system, cell or tissue in vitro, in vivo or ex vivo. Such ligand is preferably capable of generating a signal that is detectable. Any ligand (or combinations thereof) that can be used to image compounds, systems, cells or tissue in vivo and/or in vitro and that can be attached to a particles of the invention is suitable. Examples of imaging agents which can be used include enzymes, fluorescent compounds, radioisotopes, chemiluminescent compounds and bioluminescent compounds. Suitable imaging ligands may be fluorescent or near infrared (NIR) dyes such as a near infrared oligo dye. Non-limiting examples of imaging agents that can be incattached to the particles of the invention are Abz (Anthranilyl, 2-Aminobenzoyl), N-Me-Abz (N-Methyl-anthranilyl, N-Methyl-2-Aminobenzoyl), FITC (Fluorescein isothiocyanate), 5-FAM (5-Carboxyfluorescein), 6-FAM (6-Carboxyfluorescein), APC (allophycocyanin), TAMRA (Carboxytetramethyl rhodamine), Mca (7-Methoxycoumarinyl-4-acetyl), AMCA or Amc (Aminomethylcoumarin Acetate), Dansyl (5-(Dimethylamino) naphthalene-1-sulfonyl), EDANS (5-[(2-Aminoethyl)amino] naphthalene-1-sulfonic acid), Atto (e.g. Atto465, Atto488, Atto495, Atto550, Atto647), cyanine (Cy) dyes, including Cy3 (1-(5-carboxypentyl)-3,3-dimethyl-2-((1E,3E)-3-(1,3,3-trimethylindolin-2-ylidene)prop-1-en-1-yl)-3H-indol-1-ium chloride), Cy5 (1-(5-carboxypentyl)-3,3-dimethyl-2-((1E,3E,5E)-5-(1,3,3-trimethylindolin-2-ylidene)penta-1,3-dienyl)-3H-indolium chloride), including trisulfonated Cy5, and Cy7 (1-(5-carboxypentyl)-2-[7-(1-ethyl-5-sulfo-1,3-dihydro-2H-indol-2-ylidene)hepta-1,3,5-trien-1-yl]-3H-indolium-5-sulfonate), Alexa Fluor (e.g. Alexa Fluor 647, Alexa488, Alexa532, Alexa546, Alexa594, Alexa633, Alexa647), Bodipy (e.g. Bodipy® FL), Dylight (e.g. DyLight 488, DyLight 550), Trp (Tryptophan), Lucifer Yellow (ethylene diamine or 6-Amino-2-(2-amino-ethyl)-1,3-dioxo-2, 3-dihydro-1H-benzo[de]isoquinoline-5,8-disulfonic acid), a near infrared oligo dye such as Dy750, Dy751, Dy700, Dy703, Dy732, Dy734, Dy749, Dy776, Dy777, etc. and derivatives of any of these. Alternatively, the ligand may be a chelator (e.g. DOTA, DTPA, desferal and similar) to complex (radioactive) cationic metals, allowing for non-invasive imaging via SPECT or PET scans.

Alternatively, the ligand may be or comprise a therapeutic ligand. As used herein the term “therapeutic ligand” refers to an agent that can be used in therapy, e.g. for treatment purposes or vaccination purposes. Examples of therapeutic ligands that can be attached to the surface of the particles are the same as the drugs that can be entrapped in the particles of the present invention. Both the entrapped drug and the ligand on the surface are covalently attached to the polymer matrix. Examples include a macromolecule, protein, peptide, hormone, small molecule, e.g. synthetic chemical entity, or nucleic acid molecule (such as mRNA, siRNA, shRNA and DNA molecules), aptamers, or any combination thereof. Therapeutic ligands can for instance be used for therapeutic or vaccination applications. For instance, in the examples herein, the peptide SIINFEKL is conjugated to the surface of the particle for vaccination purposes. Attachement of a drug as a therapeutic ligand on the surface of the particle is particularly preferred if it is a nucleic acid, peptide or protein, the drug is optionally conjugated to the surface of the particle as a therapeutic ligand. In a particularly preferred embodiment, a therapeutic ligand is a peptide or protein. The skilled person will be able to select the appropriate therapeutic ligand based on the type of therapy, target cell and/or route of administration.

In a further aspect, the ligand may be a combination of two or more different types of ligande. For instance, a targeting moiety and an imaging agent can be combined to allow both targeting and imaging for a single particle. As another example, a targeting moiety and a therapeutic agent can be combined to allow both targeting and treatement or vaccination. Hence, preferably the ligand is selected from the group consisting of a therapeutic ligand, a targeting ligand, an imaging ligand and a combination thereof. It is further possible to attached more than one ligand, to the surface of a single nanoparticles. E.g. two or more different targeting ligands, two or more imaging ligands, two or more therapeutic ligands or a combination of one or more targeting ligands, one or more therapeutic ligands and one or more imaging ligands can be attached to the surface of the particles.

Optionally the ligand comprises an alkyne group or may be derivatised with an alkyne group.

Optionally the ligand comprises an azide group or may be derivatised with an azide group.

Optionally, more than 1, such as 2, 3, 4, or 5 alkyne groups are present on the ligand. Most optionally, the ligand comprises 1 alkyne group.

Optionally, more than 1, such as 2, 3, 4, or 5 azide groups are present on the ligand. Optionally, the ligand comprises 1 azide group.

Depending on the nature of the ligand, and the nature of the polymers, a skilled person may determine whether the polymer or ligand is better suited with a alkyne group or with an azide group.

The Azide-Alkyne Cycloaddition is a 1,3-dipolar cycloaddition between an azide and a terminal or internal alkyne to give a 1,2,3-triazole. Some cycloaddition reactions require heat or a catalyst. Optionally the reaction between the azide and alkyne is catalysed with a metal catalyst such as copper (I), ruthenium or silver. Suitably the copper (I) catalyser may be any source of copper(I) such as cuprous bromide or iodide. Optionally, the copper (I) catalyst is a mixture of copper(II) (e.g. copper(II) sulfate) and a reducing agent (e.g. sodium ascorbate) to produce Cu(I) in situ. As Cu(I) is unstable in aqueous solvents, stabilizing ligands are effective for improving the reaction outcome. Cp*RuCl(PPh₃)₂, Cp*Ru(COD) and Cp*[RuCl₄] are commonly used ruthenium catalysts, Cp* may be cyclopentadienyl (Cp) or entamehtylcyclopentadienyl. The reaction may be run in a variety of solvents, and mixtures of water and a variety of (partially) miscible organic solvents including alcohols, DMSO, DMF, tBuOH and acetone. Owing to the powerful coordinating ability of nitriles towards Cu(I), it is best to avoid acetonitrile as the solvent.

Optionally the reaction between azide and alkyne is performed without a catalyst. Optionally, the reaction between azide and alkyne is performed at room temperature Optionally the reaction between azide and alkyne is performed without a catalyst and at room temperature.

Thus the present invention is directed to a nano-particle wherein the particle is made with polymers derivatised with azide and the ligand is derivatised with alkyne.

In one aspect, the present invention provides a particle comprising a drug and a ligand wherein the particle is obtainable by a method comprising the steps of:

(i) mixing a drug comprising a reactive moiety with an aqueous solution or dispersion comprising polymer chains, at least part of these polymer chains comprising at least one azide group, the polymer chains further comprising at least one reactive moiety, capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture to cross-linking forming a polymer matrix under such conditions that simultaneous with the formation of the polymer matrix the drug is entrapped in this polymer matrix, that is in the polymeric network formed, to form a drug loaded particle;

(iv) reacting said drug entrapped particle with a ligand comprising at least one alkyne group

such that the azide group of the polymer reacts with the alkyne group of the ligand to form a triazole.

In another aspect, the present invention provides a method to produce a particle comprising a drug and a ligand, said method comprising the steps of:

i) mixing a drug comprising a reactive moiety with an aqueous solution or dispersion comprising polymer chains, at least part of these polymer chains comprising at least one azide group, the polymer chains further comprising at least one reactive moiety capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture from step (ii) to cross-linking forming a polymer matrix under such conditions that simultaneous with the formation of the polymer matrix the drug is entrapped in this polymer matrix, that is in the polymeric network formed, to form a drug loaded particle;

(iv) reacting said drug entrapped particle with a ligand comprising at least one alkyne group

such that the azide group of the polymer reacts with the alkyne group of the ligand to form a triazole.

In another aspect of the invention, the present invention provides a method to produce a particle comprising a drug and a ligand, said method comprising the steps of:

(i) providing an aqueous solution or dispersion comprising polymer chains comprising polymer chains, at least part of these polymer chains comprising at least one azide group, the polymer chains further comprising at least one reactive moiety, capable of reacting with the reactive moiety of the drug, the polymer chains further being capable of cross-linking intra- or intermolecularly; and

(ii) subjecting this mixture to conditions whereby the polymers self-assemble into particles, and,

(iii) mixing the particle from step (ii) with a solution comprising a drug such that the drug is encapsulated in the particle, and;

(iv) subjecting the particle mixture from step (iii) to cross-linking forming a polymer matrix under such conditions that simultaneous with the formation of the polymer matrix the drug is entrapped in this polymer matrix, that is in the polymeric network formed, to form a drug loaded particle;

(v) reacting said drug entrapped particle with a ligand comprising at least one alkyne group

such that the azide group of the polymer reacts with the alkyne group of the ligand to form a triazole.

Thus the present invention is directed to a nano-particle wherein the particle is made with polymers derivatised with alkyne group and the ligand is derivatised with an azide group.

In one aspect, the present invention provides a particle comprising a drug and a ligand wherein the particle is obtainable by a method comprising the steps of:

(i) mixing a drug comprising a reactive moiety with an aqueous solution or dispersion comprising polymer chains, at least part of these polymer chains comprising at least one alkyne group, the polymer chains further comprising at least one reactive moiety, capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture to cross-linking forming a polymer matrix under such conditions that simultaneous with the formation of the polymer matrix the drug is entrapped in this polymer matrix, that is in the polymeric network formed, to form a drug loaded particle;

(iv) reacting said drug entrapped particle with a ligand comprising at least one azide group

such that the azide group of the ligand reacts with the alkyne group of the polymer to form a triazole.

In another aspect, the present invention provides a method to produce a particle comprising a drug and a ligand, said method comprising the steps of:

i) mixing a drug comprising a reactive moiety with an aqueous solution or dispersion comprising polymer chains, at least part of these polymer chains comprising at least one alkyne group, the polymer chains further comprising at least one reactive moiety capable of reacting with the reactive moiety of the drug, the polymer chains further being capable of cross-linking intra- or intermolecularly; and

(ii) subjecting this mixture to conditions wherein the polymers self-assemble into particles, with the drug encapsulated in the core of the particle;

(iii) subjecting the particle mixture from step (ii) to cross-linking forming a polymer matrix under such conditions that simultaneous with the formation of the polymer matrix the drug is entrapped in this polymer matrix, that is in the polymeric network formed, to form a drug loaded particle;

(iv) reacting said drug entrapped particle with a ligand comprising at least one azide group

such that the azide group of the ligand reacts with the alkyne group of the polymer to form a triazole.

In another aspect of the invention, the present invention provides a method to produce a particle comprising a drug and a ligand, said method comprising the steps of:

(i) providing an aqueous solution or dispersion comprising polymer chains comprising polymer chains, at least part of these polymer chains comprising at least one alkyne group, the polymer chains further comprising at least one reactive moiety, capable of reacting with the reactive moiety of the drug, the polymer chains further being capable of cross-linking intra- or intermolecularly; and

(ii) subjecting this mixture to conditions whereby the polymers self-assemble into particles, and,

(iii) mixing the particle from step (ii) with a solution comprising a drug such that the drug is encapsulated in the particle, and;

(iv) subjecting the particle mixture from step (iii) to cross-linking forming a polymer matrix under such conditions that simultaneous with the formation of the polymer matrix the drug is entrapped in this polymer matrix, that is in the polymeric network formed, to form a drug loaded particle;

(v) reacting said drug entrapped particle with a ligand comprising at least one azide group

such that the azide group of the ligand reacts with the alkyne group of the polymer to form a triazole.

It should be understood that a mixture of a different drugs may be entrapped in a particle according to the present invention. Optionally, the drugs should be of a nature such that these tend to interact in a physical non-covalent manner with the polymer chains of the polymers described herein-above. In a preferred embodiment, the invention is especially useful for encapsulation of hydrophobic compounds, optionally using thermosensitive polymers.

In the particle of the present invention and/or embodiments thereof, the drug may be any drug. Examples of drugs that can be encapsulated in the particles of the present invention are a macromolecule, protein, peptide, hormone, small molecule, e.g. synthetic chemical entity, or nucleic acid molecule (such as mRNA, siRNA, shRNA and DNA molecules), aptamers, or any combination thereof. In particular in the case the drug is a nucleic acid or peptide, the drug is optionally conjugated to the surface of the particle as a therapeutic ligand. The skilled person will be able to select the appropriate drug based on the type of therapy, target cell and/or route of administration.

In a preferred embodiment of the present invention and/or embodiments thereof, the particle may comprise more than one drug. More than one drug targeting the same disease may be used, and/or drugs targeting different disease agents may be used.

Furthermore, the particle of the present invention and/or embodiments thereof, is for use as a medicine. In a preferred embodiment, the particle of the present invention and/or embodiments thereof, is for use against a disease. The invention is also related to a method of treatment using and/or administering to a subject the particle of the invention.

Optionally the disease is selected form the group consisting of cancer, infection, ophthalmological diseases, viral infection, bacterial infection, fungal infection, mucoplasma infection, parasite infection, inflammation, Dermatological diseases, Cardiovascular diseases, diseases of the central nerve system, auto-immune disease, proliferative diseases, arthritis, psychotic diseases, psoriasis, diabetes, metabolic disorders, lung diseases, respiratory diseases, pulmonary diseases, COPD, diseases of the muscoskeletal system, emphysema, edema, hormonal diseases. More specifically the particle of the present invention and/or embodiments thereof is suitable for treatment of diseases including but not limited to diseases selected from the group consisting of spinal cord injuries, heart attacks, ischaemi, arthritis, fungal infections, post operative pain, pain, non-small cell lung cancer (or cancer-small cell lung, bladder, non-Hodgkin's lymphoma, general gastrointestinal, colorectal, head and neck, breast, general solid), acute lymphocytic and acute myelogenous leukemia, breast cancer, brain cancer, general leukaemia, liver cancer, pancreas cancer, colorectal cancer, cervical cancer, general lymphoma, ovarian cancer, squamous cell cancer, general lung cancer, pancreatic cancer, bladder cancer, renal cancer, liver cancer, small cell lung cancer, stomach cancer, Hodgkin's lymphoma, non-small cell lung cancer, oesophageal cancer, adrenal cancer, melanoma, Myelodysplastic syndrome, hairy cell leukaemia, general skin, bladder, head and neck, non-small cell lung, oesophageal, ovarian, melanoma, leiomyosarcoma, biliary, breast, prostate, systemic Lupus erythematosus, mesothelioma, and/or general sarcoma.

Moreover, the particle of the present invention and/or embodiments thereof is suitable for treatment of disease including but not limited to a disease selected from the group consisting of diseases to the eyes, infectious diseases, inflammatory diseases, cancer, cardiovascular diseases, diseases from the central nervous system, autoimmune disease, and/or other diseases such as diabetes insipidus, polyuria, polydipsia, post-surgery pain and/or spinal cord injuries.

Infectious diseases may be selected from the group including bacterial infections including gram-negative infections, infections of skin, and/or fungal infections.

Inflammatory diseases may be selected from the group including rheumatoid arthritis, diabetes type I, diabetes type II, appendicitis, bursitis, colitis, cystitis, dermatitis, meningitis, phlebitis, rhinitis, tendonitis, tonsillitis, and/or vasculitis.

Cancer may be selected from the group including hormone sensitive prostate cancer, hormone sensitive breast cancer, non-small cell lung cancer, small cell lung cancer, bladder cancer, non-Hodgkin's lymphoma, general gastrointestinal cancer, colorectal cancer, head and neck cancer, breast cancer, acute lymphocytic leukaemia, acute myelogenous leukaemia breast cancer, brain cancer, leukaemia, liver cancer, testicular cancer, small cell lung carcinoma, ovarian cancer cervical cancer, squamous cell cancer, pancreatic cancer, renal cancer, stomach cancer, Hodgkin's lymphoma, oesophageal cancer, adrenal cancer, melanoma, Myelodysplastic syndrome, hairy cell leukaemia skin cancer, leiomyosarcoma, prostate cancer, systemic Lupus erythematosus, mesothelioma, and/or sarcoma.

Diseases to the eyes may be selected from the group including macular degeneration, acute postoperative endophthalmitis macular edema, and/or cataract.

Cardiovascular diseases may be selected from the group including vasoconstriction, coronary heart disease, ischaemic heart disease, coronary artery disease, cardiomyopathy, hypertensive heart disease, heart failure, cor pulmonale, cardiac dysrhythmias, inflammatory heart disease, endocarditis, inflammatory cardiomegaly, myocarditis, valvular heart disease, stroke and cerebrovascular disease, peripheral arterial disease, hypertension, and/or atherosclerosis.

Diseases from the central nervous system may be selected from the group including encephalitis, poliomyelitis, neurodegenerative diseases such as Alzheimer's disease, amyotrophic lateral sclerosis, autoimmune and inflammatory diseases such as multiple sclerosis or acute disseminated encephalomyelitis, and genetic disorders such as Krabbe's disease, Huntington's disease, and/or adrenoleukodystrophy.

Autoimmune diseases may be selected from the group including Acute disseminated encephalomyelitis (ADEM), Addison's disease, Agammaglobulinemia, Alopecia areata, Amyotrophic Lateral Sclerosis, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behçet's disease, Berger's disease, Bickerstaff's encephalitis, Blau syndrome, Bullous pemphigoid, Cancer, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis (or Idiopathic pulmonary fibrosis), Gastritis, Gastrointestinal pemphigoid, Giant cell arteritis, Glomerulonephritis, Goodpasture's syndrome, Graves' disease, Guillain-Barré syndrome (GBS), Hashimoto's encephalopathy, Hashimoto's thyroiditis, Henoch-Schonlein purpura, Herpes gestationis aka Gestational Pemphigoid, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura (See Autoimmune thrombocytopenic purpura), IgA nephropathy, Inclusion body myositis, Chronic inflammatory demyelinating polyneuropathy, Interstitial cystitis, Juvenile idiopathic arthritis aka Juvenile rheumatoid arthritis, Kawasaki's disease, Lambert-Eaton myasthenic syndrome, Leukocytoclastic vasculitis, Lichen planus, Lichen sclerosus, Linear IgA disease (LAD), Lou Gehrig's disease (Also Amyotrophic lateral sclerosis), Lupoid hepatitis aka Autoimmune hepatitis, Lupus erythematosus, Majeed syndrome, Ménière's disease, Microscopic polyangiitis, Miller-Fisher syndrome see Guillain-Barre Syndrome, Mixed connective tissue disease, Morphea, Mucha-Habermann disease aka Pityriasis lichenoides et varioliformis acuta, Multiple sclerosis, Myasthenia gravis, Myositis, Narcolepsy^{[46][47]}, Neuromyelitis optica (also Devic's disease), Neuromyotonia, Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome another form of APS, Schnitzler syndrome, Scleritis, Scleroderma, Serum Sickness, Sjögren's syndrome, Spondyloarthropathy, Still's disease see Juvenile Rheumatoid Arthritis, Stiff person syndrome, Subacute bacterial endocarditis (SBE), Susac's syndrome, Sweet's syndrome, Sydenham chorea see PANDAS, Sympathetic ophthalmia, Systemic lupus erythematosis see Lupus erythematosis, Takayasu's arteritis, Temporal arteritis (also known as “giant cell arteritis”), Thrombocytopenia, Tolosa-Hunt syndrome, Transverse myelitis, Ulcerative colitis (one of two types of idiopathic inflammatory bowel disease “IBD”), Undifferentiated connective tissue disease different from Mixed connective tissue disease, Undifferentiated spondyloarthropathy, Urticarial vasculitis, Vasculitis, Vitiligo, and/or Wegener's granulomatosis.

Other diseases may be selected from the group including diabetes insipidus, polyuria, and/or polydipsia, pruritus post-surgery pain and/or spinal cord injury including paraplegia.

The particle of the present invention and/or embodiment thereof may suitably used for several routes of administration. Suitable routes are parenteral, intravenous (i.v.), subcutaneous (s.c.,), intramuscular, intralymphatic, intraperitoneal, oral, including buccal, and sublingual, mucosal develivery, such as intra-nasal, and pulmonary, dermal such as topical, transdermal, transcutaneous.

For the purpose of clarity and a concise description features are described herein as part of the same or separate embodiments, however, it will be appreciated that the scope of the invention may include embodiments having combinations of all or some of the features described.

A skilled person will appreciate that embodiments, features and/or properties of the particle are also embodiments, features and/or properties for the methods and/or uses of the invention. A skilled person will further appreciate that embodiments, features and/or properties of the method or uses are also embodiments, features and/or properties of the particles of the invention.

Experimental Data:

Material & Methods

Docetaxel (DTX) was obtained from Phyton Biotech GmbH (Ahrensburg, Germany). N,N′-dicyclohexylcarbodiimide (DCC), 4-dimethylaminopyridine (DMAP), 4-methoxyphenol, methacrylic anhydride, ammonium acetate, formic acid, Mukaiyama's reagent (2-chloro-1-methyl-pyridinium iodide), oxone, potassium persulfate (KPS), tetramethylethylenediamine (TEMED) and trifluoroacetic acid (FFA) were obtained from Sigma Aldrich (Zwijndrecht, The Netherlands). Acetonitrile (ACN), dichloromethane (DCM), diethyl ether (DEE), N,N-dimethylformamide (DMF) and tetrahydrofuran (THF) were purchased from Biosolve (Valkenswaard, The Netherlands). Absolute ethanol and triethylamine (TEA) were purchased from Merck (Darmstadt, Germany).

L1 is 2-(2-(Methacryloyloxy)ethylthio) acetic acid

L2 is 2-(2-(methacryloyloxy)-ethylsulfinyl)acetic acid

L3 is 2-(2-(methacryl-oyloxy)ethylsulfonyl)acetic acid

Synthesis of the block copolymers Block copolymers containing a fixed hydrophilic block of monomethoxy poly(ethylene glycol) (mPEG, Mn=5000 g/mol) and a varying thermosensitive block composed of a random copolymer of N-2-hydroxypropyl methacrylamide monolactate (HPMAmLac1) and N-2-hydroxypropyl methacrylamide dilactate (HPMAmLac2) were synthesized by free radical polymerization using (mPEG5000)2-ABCPA as initiator. (C. J. Rijcken, C. J. Snel, R. M. Schiffelers, C. F. van Nostrum, W. E. Hennink, Hydrolysable core-crosslinked thermosensitive polymeric micelles: Synthesis, characterisation and in vivo studies, Biomaterials, 28 (2007) 5581-5593; D. Neradovic, C. F. van Nostrum, W. E. Hennink, Thermoresponsive polymeric micelles with controlled instability based on hydrolytically sensitive N-Isopropylacrylamide copolymers, Macromolecules, 34 (2001) 7589-7591) The comonomer feed ratio HPMAmLac1/Lac2 was kept constant at 53/47 (mol/mol), unless specified otherwise. The feed molar ratio of monomer/initiator for the “standard block copolymer” was 150 and was varied between 20 and 300 to obtain a set of alternative block copolymers of different molecular weights. To achieve this, the feed amount of total monomer (0.7 g) was kept constant while the feed amount of initiator was adjusted accordingly. In brief, HPMAmLac1, HPMAmLac2 and initiator were dissolved in ACN (450 mg of total monomer plus initiator per mL) in airtight glass vials. The reaction mixture was flushed with nitrogen for at least 10 min, heated to 70° C. and then stirred for 20-24 h. Next, the resulting block copolymers were precipitated by dropwise adding the mixture into an excess of DEE (18 mL per gram of polymer). The precipitate was filtered and dried in a vacuum oven overnight. The block copolymers were obtained as off-white solids and characterized using proton nuclear magnetic resonance (NMR) (M. Talelli, M. Iman, A. K. Varkouhi, C. J. F. Rijcken, R. M. Schiffelers, T. Etrych, K. Ulbrich, C. F. van Nostrum, T. Lammers, G. Storm, W. E. Hennink, Core-crosslinked polymeric micelles with controlled release of covalently entrapped doxorubicin, Biomaterials, 31 (2010) 7797-7804).

Derivatisation of Block Copolymer with Methacrylic Acid

A fraction (5-15 mol %) of the terminal hydroxyl groups of the lactate side chains of the synthesized block copolymer (feed molar ratio HPMAmLac1/Lac2=53/47) was derivatised with methacrylic acid (C. J. Rijcken, C. J. Snel, R. M. Schiffelers, C. F. van Nostrum, W. E. Hennink, Hydrolysable core-crosslinked thermosensitive polymeric micelles: Synthesis, characterisation and in vivo studies, Biomaterials, 28 (2007) 5581-5593) to obtain a methacrylic acid-derivatised block copolymer (referred as “MA-block copolymer”) (yield 85-95%) with a critical micelle temperature (CMT) between 5 and 15° C. The MA-block copolymers were characterized using NMR GPC and UV-Visible spectroscopy

Derivatisation of Block Copolymer with L2

A fraction (5-25 mol %) of the terminal hydroxyl groups of the lactate side chains of the synthesized block copolymer (feed molar ratio HPMAmLac1/Lac2=30/70 or 53/47) was derivatised with L2 to obtain a L2-derivatised block copolymer (referred as “L2-block copolymer”) (FIG. 1). For those L2-block copolymers that were used for micelle formation, the comonomer composition was adjusted to HPMAmLac1/Lac2=30/70 (mol/mol) to allow for a relatively lower CMT prior to derivatisation.

The carboxyl group of L2 was first activated to form a mixed anhydride 2-(2-(methacryloyloxy)ethylsulfinyl)acetic acid-pivaloyl (L2-Pv). In brief, L2 (0.46 mmol, 1 eq.) was dissolved in DCM (2.0 ml). Next, TEA (0.46 mmol, 1 eq.) was added and the reaction mixture was cooled to 0° C. Thereafter, pivaloyl chloride (0.46 mmol, 1 eq.) was added and the mixture was stirred for 1 h at 0° C. to obtain L2-Pv, which was used for the next step without further purification or analysis. To derivatise x mol % (x=5-25) of the lactate side groups with L2, block copolymer (1.50 g) was dissolved in THF (15 ml). Next, DMAP (0.03 g), L2-Pv (x % eq. compared to the terminal hydroxyl groups from lactate side groups of block copolymer) and TEA (1 eq. compared to L2-Pv) were added and the mixture was stirred at room temperature for 16 h. Thereafter, the reaction mixture was added dropwise to DEE (27 mL) to precipitate the L2-block copolymer. The precipitation, filtration and drying step were repeated once again to obtain an off-white solid (70-80% yield). The L2-block copolymers were characterized using NMR GPC and UV-Visible spectroscopy as described below. The percentage of hydroxyl end group derivatised with L2 as determined by NMR was calculated using a similar approach as utilized for MA block copolymers

Characterisation of (Derivatised) Block Copolymer by GPC and UV-VIS Spectroscopy

The molecular weights and their distributions of the synthesized (derivatised) block polymers and their distributions were determined by GPC essentially using a method reported previously (C. J. Rijcken, C. J. Snel, R. M. Schiffelers, C. F. van Nostrum, W. E. Hennink, Hydrolysable core-crosslinked thermosensitive polymeric micelles: Synthesis, characterisation and in vivo studies, Biomaterials, 28 (2007) 5581-5593) except that a PFG 5 μm Linear S column (Polymer Standards Service, Germany) was used.

The CMT of the synthesised (derivatised) block copolymers in aqueous solutions was recorded on a UV-2450 spectrophotometer (Shimadzu, Japan). Prior to measurement, the block copolymers were dissolved overnight at 4° C. in ammonium acetate buffer (150 mM, pH 5.0) at a concentration of 2 mg/mL. The wavelength and slit width were 650 nm and 2 nm, respectively. The ramping heating rate was 1° C./min and the interval of recording of the scattering intensity was 0.2° C. The onset on the X-axis, obtained by extrapolation of the absorbance-temperatures curve to the baseline, was considered as the CMT of the block copolymer.

Synthesis and Analysis of DTX Derivatives

Synthesis of DTXL1

L1 was conjugated to the hydroxyl group at the C-2′ position of DTX to obtain DTXL1 (FIG. 2). In brief, L1 (24.75 mmol) was dissolved in DCM (1000 mL) and stirred at 750 rpm. Next, DMAP (59.41 mmol), DTX (24.75 mmol) and Mukaiyama's reagent (29.70 mmol) were added and the mixture was placed in a pre-heated oil bath and stirred at 40° C. for 1 h to obtain a yellow solution. Next, the mixture was cooled down to room temperature and water (450 mL) was added to yield a two-phase system. The aqueous layer was extracted with DCM (300 mL) and the combined organic layers were dried with Na₂SO₄, filtered and evaporated in vacuo to obtain a yellow oil. The resulting oil was purified by column chromatography (heptane/ethyl acetate (4/1 to 1/1)) to obtain DTXL1 as a white solid (71% yield) with purity of >95%.

Synthesis of DTXL2

L2 was conjugated to the hydroxyl group at the C-2′ position of DTX to obtain DTXL2 (FIG. 2) using the same synthesis and purification methods as described above (59% yield) with purity of >95%.

Synthesis of DTXL3

The sulfur atom in the linker segment of DTXL1 was oxidized to obtain DTXL3 (FIG. 2). In brief, DTXL1 (17.10 mmol) was dissolved in ACN/water (60%/40% (v/v)) mixture (213 mL) and stirred at room temperature for 30 min to obtain a homogeneous solution. Thereafter, oxone (22.23 mmol) was added and the resulting mixture was stirred at room temperature for 2 d. Next, water (170 mL) was added to separate the layers. The organic layer was collected and the aqueous layer was extracted twice with ethyl acetate (200 mL). The combined organic layers were washed with water (100 mL), dried with Na₂SO₄, filtered and evaporated in vacuo. The obtained solid was purified by column chromatography (heptane/ethyl acetate (3/1 to 1/3)) to obtain a white solid (80% yield) with purity of >95%.

Synthesis of DTX(L2)₂

Two L2 linkers were conjugated to the hydroxyl groups at the C-2′ and C-7 positions of DTX, respectively, to obtain DTX(L2)2 (FIG. 2). In brief, DTX (2.5 mmol), L2 (5.0 mmol), Mukayama's reagent (6.20 mmol) and DMAP (12.4 mmol) were dissolved in DCM (83 mL) and stirred at 40° C. for 1 h. Next, the reaction mixture was washed with brine and water and the organic layer was dried with MgSO₄, filtered and evaporated in vacuo. The oily residue was purified using flash chromatography (ethyl acetate/n-hexane (9/1)) to obtain DTX(L2)₂ as an amorphous white solid (23% yield) with purity of >90%.

Analysis of DTX Derivatives

Proton NMR spectra of the DTX derivatives were recorded using a Gemini 300 MHz spectrometer (Varian Associates Inc. NMR Instruments, Palo Alto, Calif.). The ¹H NMR spectra of DTX derivatives were obtained in DMSO-d6 solvent.

The molecular mass of DTX derivatives was determined using electrospray ionization mass spectrometry (ESI-MS) on a Shimadzu liquid chromatography-mass spectrometry (LC-MS) QP8000 in positive ion mode. A Gemini®3 μm C18 column (150×3 mm) (Phenomenex) was used with a gradient from 100% eluent A (95% H2O/5% ACN/0.1% trifluoroacetic acid) to 100% B (5% H2O/95% ACN/0.1% trifluoroacetic acid) in 1 h with a flow of 1 mL/min and UV-detection at 253 nm.

The purity of DTX derivatives was determined by ultra-performance liquid chromatography (UPLC) (Waters, USA) equipped with a UV-detector (TUV, Waters). An Acquity HSS T3 1.8 μm column (50×2.1 mm) (Waters) was used for an isocratic run of 20 minutes (mobile phase: 0.1% formic acid in H₂O) with a flow of 0.7 mL/min and UV-detection at 227 nm. DTX derivative standards dissolved in ACN/water (70%/30% (v/v)) mixture were used to prepare a calibration curve (linear between 0.5 and 100 μg/mL).

Particle Size Distribution

The particle size of core-cross-linked polymeric micelles (CCL-PMs) was measured by dynamic light scattering (DLS) using a Malvern ALV/CGS-3 Goniometer. The viscosity and refractive index of water at 25° C. were used for the all measurements. DLS results are given as a z-average particle size diameter (Zave) and a polydispersity index (PDI).

Analysis of DTXLx-CCL-PMs by UPLC

The contents of released DTX, and total (i.e. released and entrapped) DTX in DTXLx-CCL-PMs were determined by UPLC. To determine the contents of released DTX, the micellar dispersion was diluted 10 times with a mixture of ACN/water (70%/30% (v/v)) mixture, and next 7 μL of the resulting mixture was injected into UPLC equipped with a UV-detector (TUV, Waters). An Acquity HSS T3 1.8 μm column (50×2.1 mm) (Waters) was used for an isocratic run of 6 min (mobile phase: 50% H2O/50% ACN/0.1% formic acid) with a flow of 0.8 mL/min and UV-detection at 227 nm. DTX and DTX derivative standards dissolved in ACN/water (70%/30% (v/v)) mixture were used to prepare a calibration curve (linear between 0.5 and 100 μg/mL).

The total content of DTX in DTXLx-CCL-PMs was measured indirectly by quantifying the content of benzoic acid (the final degradation product of DTX) as described by Q. Hu, C. J. Rijcken, R. Bansal, W. E. Hennink, G. Storm, J. Prakash, Complete regression of breast tumour with a single dose of docetaxel-entrapped core-cross-linked polymeric micelles, Biomaterials, 53 (2015) 370-378.

The drug entrapment efficiency (EE) was calculated using the UPLC data as follows:

$$EE = \frac{Amount of entrapped DTX}{Amount of DTX equiv . added} \times 100 \%$$

In Vitro Drug Release from DTXLx-CCL-PMs

The in vitro release of DTX from DTXLx-CCL-PMs was measured in phosphate buffered saline (pH 7.4) at 37° C. In brief, DTX-CCL-PMs were diluted 20 times in phosphate buffer (100 mM, pH 7.4, supplemented with 15 mM NaCl) containing 1% (v/v) polysorbate 80 (to solubilize the released DTX). The mixture was incubated at 37° C. and samples were collected at different time points and analyzed for released DTX and for 7-epi-DTX (the known epimer of DTX contents using UPLC. The concentrations of released DTX and 7-epi-DTX were determined by injecting 7 μL of the mixture into a UPLC system. An Acquity HSS T3 1.8 μm column (50×2.1 mm) (Waters) was used with a gradient from 100% eluent A (70% H₂O/30% ACN/0.1% formic acid) to 100% B (10% H₂O/90% ACN/0.1% formic acid) in 11 minutes with a flow of 0.7 mL/min and UV-detection at 227 nm. DTX standards dissolved in mixture of ACN/water (70%/30% (v/v)) mixture were used to prepare a calibration curve (linear between 0.5 and 100 μg/mL) to determine the concentration of released DTX and of 7-epi-DTX. To calculate the percentage of actual DTX, only DTX and 7-epi-DTX (which together constitute>90% of the total peak area in the chromatogram) were taken into account, and so not the other degradation products of DTX that are generated in time under physiological conditions due to the hydrolytic instability of DTX

$$\% Actual DTX = \frac{Amount of DTX + Amount of 7 \text{-} epi \text{-} DTX}{Amount of total DTX} \times 100 \%$$

In Vitro Degradation Profile of CCL-PMs

The degradation kinetics of empty CCL-PMs based on block copolymers derivatised with either methacrylic acid (5 or 10 mol % of the hydroxyl end group of lactate side chain) or L2 (5 or 10 mol % of the hydroxyl end group of lactate side chain) were studied in vitro. In brief, the empty CCL-PMs dispersions were diluted 5 times with phosphate buffer (100 mM, pH 7.4, supplemented with 15 mM NaCl) or borate buffer (100 mM, pH 9.4) and incubated at 37° C. and 60° C., respectively. The Zave and PDI of these incubated dispersions were monitored using DLS. In addition, the derived count rate (DCR,), in kilo counts per second (kcps)), was also recorded during DLS measurements. The DLS measurement was terminated when the DCR decreased to <100 kcps.

Results:

High Drug Loading Capacity

Table 1 indicates the loading capacity (LC) of the polymer particles as a function of feed polymer and feed of drug. It can be seen that the size of the particle (Zave) and the polydispersity (DPI) are not influenced. A loading capacity of over 20% can be obtained. FIG. 3 shows the high loading capacity and drug entrapment efficiency at a drug feed of about 4 mg/ml.

TABLE 1

| | Feed DTX | | | | |
|---|---|---|---|---|---|
| Feed polymer | equiv. | Ratio Drug: | | Z-ave | |
| (mg/mL) | (mg/mL) | polymer | LC % | (nm) | PDI |
| 20 | 2 | 0.1 | 12 | 65 | 0.02 |
| 17.5 | 4 | 0.24 | 23 | 74 | 0.08 |
| 20 | 4 | 0.2 | 21 | 71 | 0.08 |
| 25 | 4 | 0.16 | 18 | 71 | 0.07 |
| 30 | 4 | 0.13 | 15 | 70 | 0.07 |
| 35 | 4 | 0.11 | 13 | 71 | 0.08 |
| 40 | 4 | 0.1 | 11 | 70 | 0.07 |

FIG. 4 shows that the release of drugs is also not signifcantly changed when higher drug loading capacity particles are used.

It was found that the high drug entrapment (loading capacity) also doubled the entrapment efficiency. In addition, the inventors unexpected found that also small particles have a large loading capacity to entrap large drug quantities

Table 2 shows that the type of linker; does not change size of particle indicating that different release profiles may be obtained while still having the same size of particle.

CriPec docetaxel: only difference is the type of linker used to derivatise docetaxel—all other properties are equal.

TABLE 2

| DTX- | Z-Ave | | |
|---|---|---|---|
| linker | (nm) | PDI | EE % |
| DTXL1 | 74 | 0.08 | 63 |
| DTXL2 | 67 | 0.03 | 70 |
| DTXL3 | 69 | 0.08 | 66 |
| DTX(L2)2 | 71 | 0.09 | 66 |

TABLE 3

| Linker | | Drug release kinetics |
|---|---|---|
| type | # linker | (pH 7.4, 37° C.) |
| L1 | 1 | <10% in 8 d |
| L2 | 1 | <15% in 8 d |
| L2 | 2 | T_{1/2} = 3.1 d |
| L3 | 1 | T_{1/2} = 1.6 d |

Table 3 and FIG. 5 shows that the drug release kinetic is not changed much upon use of different linkers.

Conjugation of Cyclic RGDfk-BCN to N₃-Nanoparticle

FIG. 7 indicates the conjugation of a cyclic RGDfk-BCN to N₃-CriPec nanoparticle.

Synthesis of BCN-cRGDfK

Cyclic RGDfK peptide was prepared and cyclized using solid support peptide synthesis. After cleavage the crude peptide was purified by HPLC. Bicyclononyne-RGDfk (BCN-RGDfK) was prepared by reacting purified cRGDfK (cyclic Arg-Gly-Asp peptide) with BCN-p-Nitrophenyl carbonate, followed by HPLC purification.

Synthesis of 5% N₃ CriPec Nanoparticles

5% N₃ CriPec empty nanoparticles (NPs) composed of 95 w % plain CriPec block copolymer (mPEG-b-HPMAmDPₙ) and 5 w % azide block copolymer (N₃-PEG-b-HPMAmDPₙ) are synthesised. This azide block copolymer is synthesized as described above, with the exception that azide-PEG5000 is used as starting material as compared to mPEG5000. The synthesis of the nanoparticles goes as amongst others described in Q. Hu, C. J. Rijcken, R. Bansal, W. E. Hennink, G. Storm, J. Prakash, Complete regression of breast tumour with a single dose of docetaxel-entrapped core-cross-linked polymeric micelles, Biomaterials, 53 (2015) 370-378.

The obtained 5% N₃ CriPec NPs are purified by means of tangential flow filtration (TFF) and concentrated (if necessary) for conjugation.

Synthesis of RGD CriPec NPs

N₃ CriPec NPs are reacted with the amounts of BCN-cRGDfK as indicated in aqueous buffer overnight at room temperature. The depletion of BCN-cRGDfK is monitored by UPLC over time and conjugation is followed by 15N NMR. The NMR spectrum is shown in FIG. 8; the absence of any free azide molecules and the presence of typical triazol peaks demonstrated that covalent conjugation between RGD and CriPec empty was indeed generated. Next, RGD CriPec NPs are purified by means of TFF to remove non-conjugated BCN-cRGDfK (if any) and other impurities.

The equivalent is the amount of alkyne containing ligand relative to azide.

TABLE 4
monitoring the RGD conjugation to
N3 CriPec nanoparticles via UPLC

| | % | % | | |
|---|---|---|---|---|
| | Conversion | Ligand | % Azide | % Free |
| | t = 1 w | t = 1 w | Conversion | Azide |
| Blank | 0 | — | — | — |
| Negativecontrole, | 1.2 | — | — | — |
| 100% mPEG | | | | |
| CriPec empty. | | | | |
| 100% Azide, 1 eq | 78.1 | 78 | 78 | 22 |
| 20% Azide, 1 eq | 71.4 | 14.3 | 72 | 5.7 |
| 10% Azide, 1 eq | 70.1 | 7.0 | 70 | 3.0 |
| 10% Azide, 0.8 eq | 67.4 | 5.4 | 54 | 4.6 |
| 10% Azide, 0.4 eq | 70.3 | 2.8 | 28 | 7.2 |
| 10% Azide, 0.2 eq | 76.6 | 1.5 | 15 | 8.5 |

65 nm sized N3 CriPec NP

Table 4 shows that conjugation of ligand has a high efficiency
Table 5 and 6 show that there is no effect of the surface conjugation on the particle size

TABLE 5

| Description | Z_{Ave} (nm) | PDI |
|---|---|---|
| 1.4% RGD CriPec empty | 41 | 0.10 |
| 0% RGD CriPec DOX | 41 | 0.14 |
| 0.5% RGD CriPec DOX | 43 | 0.14 |
| 1.5% RGD CriPec DOX | 43 | 0.14 |
| 1.8% RGD CriPec DOX | 43 | 0.16 |

DOX = doxorubicin

TABLE 6

| Description | Z_{Ave} (nm) | PDI |
|---|---|---|
| CriPec empty containing rhodamine | 37 | 0.16 |
| 1% RGD CriPec empty containing | 41 | 0.14 |
| rhodamine | | |
| 5% RGD CriPec empty containing | 43 | 0.14 |
| rhodamine | | |

Table 6 shows CriPec nanoparticles (NP) with rhodamine conjugated to polymer, thus entrapped in the nanoparticle. It can be seen that the particle does not change upon conjugation of a targeting ligand

Table 7 shows that that the conjugation of ligand to the surfaces has no effect on the entrapped drug. There is no burst release (BR), i.e. after conjugation no free doxorubicine is measurable.

TABLE 7

| | | | Total | % BR | | |
|---|---|---|---|---|---|---|
| Batch code and | Z-Ave | | DOX | | DOX- | |
| description | d · nm | PdI | (ug/mL) | DOX | MA | % LC |
| NP262C; RGD | 41 | 0.10 | 0 | <1 | <1 | NA |
| (1 eq.) CriPec | | | | | | |
| empty | | | | | | |
| NP263C; 0% RGD | 41 | 0.14 | 1000 | <1 | 0.12 | 6.1 |
| CriPec doxorubicin | | | | | | |
| NP264C; 0.5% RGD | 43 | 0.14 | 1000 | <1 | <1 | 5.9 |
| (0.1 eq.) CriPec | | | | | | |
| doxorubicin | | | | | | |
| NP265C; 1.5% RGD | 43 | 0.14 | 1000 | <1 | <1 | 5.8 |
| (0.5 eq.) CriPec | | | | | | |
| doxorubicin | | | | | | |
| NP266C; 1.8% RGD | 43 | 0.16 | 1000 | <1 | <1 | 5.9 |
| (1 eq.) CriPec | | | | | | |
| doxorubicin | | | | | | |

% BR = burst release.

Table 7 shows that particles containing targeting ligands do not have changed properties with respect to loading capacity, burst release, size and polydispersity. This means that further optimisation of the administration is not necessary.

Particle Degradation

The linker type and density determines the degradability of the particle. One may obtain full disintegration within weeks or months depending on the linker and thus the nanoparticle can be designed as desired.

TABLE 8

| Crosslink | Crosslink | Full nanoparticle degradation | |
|---|---|---|---|
| type | density | kinetics (pH 7.4, 37° C.) | |
| MA | 5% | Ca. 200 | d |
| MA | 10% | Ca. 400 | d |
| L2 | 5% | Ca. 30 | d |
| L2 | 10% | Ca. 30 | d |

Table 8 and FIG. 6 show full degradation profile of the nanoparticle depending on linker type and density.

Pharmakinetic Data Nanoparticles

Female NCr nu/nu mice age 8 to 12 weeks were injected with nanoparticles with entrapped doxorubicin with and without RGD. Particles were 35 or 65 nm:

Group 1=CriPec doxorubicin 35 nm in 180 mM HEPES, pH 7.4
Group 2=CriPec doxorubicin 65 nm in 180 mM HEPES, pH 7.4
Group 3=RGD CriPec doxorubicin 35 nm in 180 mM HEPES, pH 7.4
Group 4=RGD CriPec doxorubicin 65 nm in 180 mM HEPES, pH 7.4
Single dose intravenous injection at a dose of 12 mg/kg. Blood sampling: 5 min, 1, 2, 8, 24 and 48 h after injection (table 9). Measurement of total and released doxorubicin using validated methods.

TABLE 9

| | | Post single dose sample collection time points | | | | | | |
|---|---|---|---|---|---|---|---|---|
| | | 0.08 h | | | | | | |
| Group | Animals | (5 min) | 1 h | 2 h | 8 h | 24 h | 48 h | total |
| 1 | 1-10 | 1-10 | 1, 2, 3, 4 | 5, 6, 7, 8 | 1, 2, 3, 4 | 5, 6, 9, 10 | 7, 8, 9, 10 | 30 |
| 2 | 1-10 | 1-10 | 1, 2, 3, 4 | 5, 6, 7, 8 | 1, 2, 3, 4 | 5, 6, 9, 10 | 7, 8, 9, 10 | 30 |
| 3 | 1-10 | 1-10 | 1, 2, 3, 4 | 5, 6, 7, 8 | 1, 2, 3, 4 | 5, 6, 9, 10 | 7, 8, 9, 10 | 30 |
| 4 | 1-10 | 1-10 | 1, 2, 3, 4 | 5, 6, 7, 8 | 1, 2, 3, 4 | 5, 6, 9, 10 | 7, 8, 9, 10 | 30 |
| total | 40 | 40 | 16 | 16 | 16 | 16 | 16 | 120 |

FIGS. 9 and 10 show a comparison of 35 nm and 65 nm unconjugated or 1% RGD conjugated nanoparticles. It reveals a similar pharmacokinetic profile for both total and released doxorubicin which indicated that clearance of small (35 nm) and large (65 nm) of targeted and untargeted nanoparticles is similar in this mouse model.

Synthesis of Desferal CriPec Empty

CriPec nanoparticles with desferal attached to their surface were prepared by overnight conjugation of 5% N₃ CriPec nanoparticles (prepared as described above) and 5 equivalents of desferal-BCN in 20 mM ammonium acetate buffer (pH 5) with DMSO (see FIG. 11A). The buffer was replaced by 100 nM sodium acetate-d3 with 0.2 mM EDTA and the reaction between the N₃ CriPec nanoparticles and desferal-BCN was followed by ¹⁵N NMR (HMBC ¹⁵N-¹H). The NMR spectrum is shown in FIG. 11B; the absence of any free azide molecules and the presence of typical triazol peaks demonstrated that covalent conjugation between desferal and CriPec empty was indeed generated. DLS analysis of desferal CriPec empty depicted that the size and PDI are 48 nm and 0.21, respectively, i.e. no change in particle size upon desferal conjugation.

Synthesis of Dy751 CriPec Nanoparticles

CriPec nanoparticles with Near Infrared (NIR) Dye Dy 751 (Dyomic) attached to their surface were prepared by overnight conjugation of 1:1 BCN-DY751 to 5% N₃ CriPec empty (prepared as described above) in ammonium acetate buffer (pH 5) with DMSO (see FIG. 12). The reaction was followed by tracking the level of unconjugated BCN-DY751 by UPLC. Non-functionalized nanoparticles (i.e. lacking the azide-group) were used as a control. After conjugation, the DY751 CriPec empty were purified by tangential flow filtration and characterised. Their features are listed below in table 10.

TABLE 10

| | | | | | Polymer | |
|---|---|---|---|---|---|---|
| | | | Z-ave | | content | Free |
| Test item | Polymer composition | Dispersed in | (nm) | PDI | (mg/mL) | NIR-BCN |
| 1.6% DY751 | 5 w % L1 N₃-PEG- | 20 mM ammonium | 43 | 0.23 | 9 | not |
| CriPec ® | b-pHPMAmDPn | acetate pH 5 + | | | | detetable |
| empty | 95 w % L1 mPEG- | 130 mM NaCl | | | | |
| | b-pHPMAmDPn | | | | | |

The biodistribution of these Dy751 micelles was monitored in tumour bearing mice. Nude mice were injected orthotopically on day 0 with 200.000 IRFP (Infra Red Fluorescent Protein; exc 680 nm) labeled 4T1 breast cancer cells. Upon confirmation of metastasis by CT scan animals were i.v. injected on day 42 with NIR labeled nanoparticles for in vivo/ex vivo Fluorescence Molecular Tomography; FMT. Animals were monitored at 15 min, 4, 24 and 72 hrs post injection by CT/FMT.

This technique allowed for the detection of spontaneous metastatic colonization, and moreover the increasing CriPec® nanoparticle accumulation in primary tumour and metastases could non-invasively be monitored over time (FIG. 13).

Synthesis of RGD CriPec AHA1 nanoparticles

CriPec nanoparticles targeted with peptide RGD on their surface and with covalently entrapped ds siRNA AHA1 were prepared. The biological target of AHA1 is Hsp90 chaperone Aha1. A hydrolysable linker (an acid-sensitive or cleavable under physiological conditions) is attached to the ds siRNA AHA1, whereby the reactive moiety is a 5′-amino.

Nanoparticles with covalently entrapped ds siRNA AHA1 containing an acid-sensitive linker (AHA1L7) were prepared by adding AHA1L7 dropwise to a mixture of 95% L2-derivatised mPEG-b-p(HPMAmDP1DP2) polymer and L2-derivatised 5% N₃-PEG-b-p(HPMAmDP1DP2) polymer to obtain 5% N₃ CriPec AHA1 nanoparticles.

The siRNA-linker is hereby covalently entrapped within the polymeric matrix of the CriPec nanoparticles as a result of crosslinking of the reactive (methacrylate) moieties of both siRNA linker and polymer in the presence of TEMED and KPS (similarly as described above, and according to Q. Hu, C. J. Rijcken, R. Bansal, W. E. Hennink, G. Storm, J. Prakash, Complete regression of breast tumour with a single dose of docetaxel-entrapped core-cross-linked polymeric micelles, Biomaterials, 53 (2015) 370-378); followed by RGD conjugation to the surface.

RGD was attached to the nanoparticles by conjugating BCN-cRGDfK to the 5% N₃ CriPec AHA1 nanoparticles as described above for RGD CriPec nanoparticles.

The release of drug (AHA1) was determined as described above for DTXLx-CCL-PMs.

FIG. 14A shows that the presence of targeting ligand RGD peptide at the surface of the nanoparticles does not affect release kinetics.

FIG. 14B shows that under slightly acidic conditions (pH 5.5) selective release of AHA1 from the nanoparticles with covalently entrapped ds siRNA AHA1 containing a acid sensitive linker (L7) occurs as compared to pH7.4. This figure further shows that the presence of the targeting ligand (RGD) does not affect the release profile under these conditions.

Synthesis of SIINFEKL CriPec Empty

CriPec nanoparticles without any drug entrapped (i.e. CriPec empty) with ovalbumine peptide SIINFEKL (OVA residues 257-264) attached to their surface were prepared, for application as vaccination agent. First, SIINFEKL was derivatised with a BCN compound via conjugation of BCN-PEG4-NHS to the terminal NH2 of SIINFEKL (FIG. 15A).

Thereafter, the SIINFEKL-BCN was conjugated to 5% N3 CriPec empty in pH 5 buffer and the conjugation conversion was monitored by determining the level of unreacted SIINFEKL-BCN via UPLC (FIG. 15B). A negative control of non-functionalised (azide free CriPec empty) ran alongside to differentiate between actual conjugation and potential physical adsorption. Similarly as above for desferal and RGD, the conjugation was also monitored via 15N NMR and full conversion of all azide moieties was demonstrated.

The SIINFEKL CriPec empty was thereafter characterised, as described herein before, with the following results shown in table 11:

TABLE 11

| Parameter | Method | Target Specs | | Result |
|---|---|---|---|---|
| Appearance | Visual | Slightly opalescent and | | complies |
| | | homogenous fluid | | |
| Particle size | Malvern DLS | 50-75 | nm | 58 nm |
| PDI | Malvern DLS | ≤0.2 | | 0.2 |
| % of SIINFEKL | UPLC | 3-5% | w/w | 2.5-5% |
| on surface | | | | |
| Free SIINFEKL | UPLC | <2% | w/w | Not Detected |
| Free SIINFEKL- | UPLC | <2% | w/w | <1% |
| linker | | | | |
| Polymer content | UPLC | 30 | mg/mL | 30 |

1-4. (canceled)

5. A method to produce a particle comprising a drug or a ligand or both, said method comprising the steps of:
(i) subjecting an aqueous solution or dispersion comprising polymer chains being capable of cross-linking intra- or intermolecularly to conditions wherein the polymers self-assemble into particles; and
(ii) subjecting the particles to cross-linking forming a polymer matrix;
(a) wherein the particles contain a drug present in an amount of at least 10% of said particle and said polymer chains comprise at least one first reactive moiety that reacts with a second reactive moiety of the drug and prior to or subsequent to subjecting said aqueous solution or dispersion to conditions wherein the polymers self-assemble into particles, mixing said solution or dispersion with a drug containing said second reactive moiety to obtain said particles comprising at least 10% of said drug; or
(b) wherein the particles comprise a ligand wherein said polymer chains comprise an azide group or an alkyne group and said method further comprises reacting said particles with a ligand comprising at least one alkyne group when the polymer chain comprises an azide group, or
reacting said particles with a ligand comprising at least one azide group when the polymer chain comprises an alkyne group,
such that the azide group reacts with the alkyne group to form a triazole bond to obtain said particles comprising a ligand; or
(c) wherein the particles comprise a drug and a ligand wherein said polymer chains comprise at least one first reactive moiety that reacts with a second reactive moiety of the drug and said method further comprises mixing said solution or dispersion with a drug containing said second reactive moiety prior to or subsequent to subjecting said aqueous solution or dispersion to conditions wherein the polymers self-assemble into particles, and wherein said polymer chains comprise an azide group or an alkyne group and said method further comprises reacting said particles with a ligand comprising at least one alkyne group when the polymer chain comprises an azide group, or
reacting said particles with a ligand comprising at least one azide group when the polymer chain comprises an alkyne group,
such that the azide group reacts with the alkyne group to form a triazole bond to obtain said particles comprising a drug and ligand.

6. The method of claim 5, wherein the polymer chains are di- or triblock copolymers.

7. The method of claim 6, wherein part of the block copolymers comprise a thermosensitive (co)polymer.

8. The method of claim 7, wherein the thermosensitive polymer is selected from (co)polymers based on hydrophobically modified esters of N-hydroxyalkyl-(meth)acrylamide or N-(meth)acryloyl amino acids and optionally wherein the thermosensitive polymer chains include also monomers derived from N-isopropylacrylamide and/or alkyl-2-oxazalines.

9. (canceled)

10. The method of claim 5, wherein the polymer chains contain functional groups, such as (co)polymers of N-hydroxyalkyl methacrylamide-oligolactates, including (oligo)lactate esters of HPMAm (hydroxypropyl methacrylamide) or HEMAm (hydroxyethylmethacrylamide).

11. (canceled)

12. The method of claim 6, wherein the block polymers comprise PEG.

13. The method of claim 12, wherein the azide group or the alkyne group is attached to PEG.

14. The method of claim 5, wherein the polymers comprise micelle, hydrogel, micro particle and/or coating forming polymers, preferably based on thermosensitive polymers.

15. The method of claim 5, wherein the drug is attached to the polymer matrix via a degradable linker.

16. (canceled)

17. The method of claim 5 wherein the ligand is a therapeutic ligand, a targeting ligand and/or an imaging ligand.

18. A particle obtainable by the method of claim 5.

19. (canceled)

20. The particle of claim 18, wherein said particle comprises an imaging ligand attached to the surface.

21-22. (canceled)

23. A method of treatment comprising administering to a subject the particle of claim 18.

24. The method of claim 23, wherein said particle comprises an imaging ligand attached to the surface and said method further comprises diagnosis.

25. The method of claim 5, wherein the particles contain a drug present in an amount of at least 10% of said particle and said polymer chains comprise at least one first reactive moiety that reacts with a second reactive moiety of the drug and prior to or subsequent to subjecting said aqueous solution or dispersion to conditions wherein the polymers self-assemble into particles, mixing said solution or dispersion with a drug containing said second reactive moiety to obtain said particles comprising at least 10% of said drug.

26. The method of claim 5, wherein the particles comprise a ligand wherein said polymer chains comprise an azide group or an alkyne group and said method further comprises reacting said particles with a ligand comprising at least one alkyne group when the polymer chain comprises an azide group, or
reacting said particles with a ligand comprising at least one azide group when the polymer chain comprises an alkyne group,
such that the azide group reacts with the alkyne group to form a triazole bond to obtain said particles comprising a ligand.

27. The method of claim 5, wherein the particles comprise a drug and a ligand wherein said polymer chains comprise at least one first reactive moiety that reacts with a second reactive moiety of the drug and said method further comprises mixing said solution or dispersion with a drug containing said second reactive moiety prior to or subsequent to subjecting said aqueous solution or dispersion to conditions wherein the polymers self-assemble into particles and wherein said polymer chains comprise an azide group or an alkyne group and said method further comprises reacting said particles with a ligand comprising at least one alkyne group when the polymer chain comprises an azide group, or
reacting said particles with a ligand comprising at least one azide group when the polymer chain comprises an alkyne group,
such that the azide group reacts with the alkyne group to form a triazole bond to obtain said particles comprising a drug and ligand.

28. The method of claim 25 wherein at least part of said polymer chains comprise an azide group or an alkyne group.
